(19) United States
(12) Patent Application Publication
(10) Pub. No.: US 2025/0387495 A1
(43) Pub. Date: Dec. 25, 2025

(54) A METHOD FOR PREPARING A POLYETHYLENE GLYCOL ALDEHYDE DERIVATIVE

(71) Applicant: XIAMEN SINOPEG BIOTECH CO., LTD., Xiamen, Fujian (CN)

(72) Inventors: Sheng LIN, Xiamen (CN); Ailan WANG, Xiamen (CN); Qi ZHU, Xiamen (CN); Wengui WENG, Xiamen (CN); Chao LIU, Xiamen (CN)

(73) Assignee: XIAMEN SINOPEG BIOTECH CO., LTD., Xiamen, Fujian (CN)

(21) Appl. No.: 18/879,062

(22) PCT Filed: Jun. 30, 2023
(86) PCT No.: PCT/CN2023/105409

§ 371 Date: Dec. 26, 2024

(30) Foreign Application Priority Data
Aug. 26, 2022 (CN) 202211032645.4

(51) Int. Cl. A61K 47/60; C08G 65/26

(52) U.S. Cl. CPC A61K 47/60; C08G 65/2609

(57) ABSTRACT

An improved method for preparing polyethylene glycol acetal and aldehyde derivative, and the polyethylene glycol acetal derivative is represented by formula (1) or formula (2) used to preparing a series of linear and nonlinear polyethylene glycolaldehyde derivatives with a single aldehyde group or multiple aldehyde groups, and with high yield, high purity and high terminal substitution, using a small-molecule cyclic acetal derivative and a polyethylene glycol derivative as raw materials in the presence of a base reagent derivative.

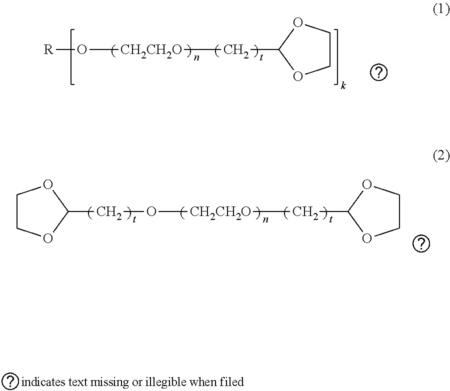

### TECHNICAL FIELD

The present application relates to the field of macromolecular synthetic chemistry, especially to a method for preparing a polyethylene glycol acetal derivative or a polyethylene glycol aldehyde derivative.

### BACKGROUND

PEGylation is one of the most important means of modification of drugs or bio-related substances, and the modified drug molecules will acquire many excellent properties of polyethylene glycol (e.g., hydrophilicity, flexibility, anticoagulant properties, etc.). Wherein, the polyethylene glycol aldehyde modifiers with an aldehyde as the terminal group are highly important modifiers in the field of proteins, which have the advantages of high selectivity and high activity retention. The aldehyde group has a certain selectivity for the N-terminus of protein, because the PKa of the N-terminal amino group of a protein is lower than that of the side chain amine group. When the side chain amino group is protonated at a certain pH and loses the ability to perform nucleophilic attack on aldehyde groups, the N-terminal amino group remains unprotonated and retains its nucleophilic attack ability. Aldehyde groups can also form a Schiff base with the N-terminal amino group of a protein, which, after reduction, results in a stable imine linkage while preserving the positive charge of the amino group, playing an important role in maintaining the structure and activity of proteins.

Prior art U.S. Pat. No. 6,465,694B1 discloses that polyethylene glycol aldehyde can be obtained by oxidizing the terminal hydroxyl group of polyethylene glycol, e.g., by adding oxygen to a mixture of PEG and a catalyst to oxidize the —CH₂OH group to —CHO. However, under most oxidizing conditions, the PEG chain tends to decompose, leading to inefficient conversion of the terminus of the PEG chain. Polyethylene glycol aldehyde can also be prepared using acetals, obtains by introducing a linear acetal group at the end of the PEG chain and subsequent hydrolyzing, but the linear acetal used as a starting material in this method is unstable and may produce by-products. Specifically, CN1763122A discloses that the alkali-catalyzed reaction between 3-hydroxypropanal diethyl acetal and PEG methanesulfonate will produce a large amount of unstable PEG vinyl ether in the side reaction, with a reaction yield of less than 85%-90%; additionally, intermolecular coupling may occur, leading to low purity of the resulting polyethylene glycol aldehyde product, thus affecting the modification efficiency of polyethylene glycol aldehyde on proteins and other drugs. CN102037056A discloses a method of preparing a high-purity polyethylene glycol aldehyde, but it is not suitable for commercialization because of the cumbersome reaction steps.

Therefore, it is necessary to improve the existing preparation methods and develop a method with fewer reaction steps to obtain the polyethylene glycol aldehyde derivatives with high terminal substitution rate, high yields, and high purity.

### SUMMARY

To achieve the above objectives, the present application provides a method for preparing the polyethylene glycol acetal derivative represented by formula (1) or formula (2), and the embodiment is as follows:

A method for preparing a polyethylene glycol acetal derivative, wherein the structure of the derivative is represented by formula (1) or formula (2):

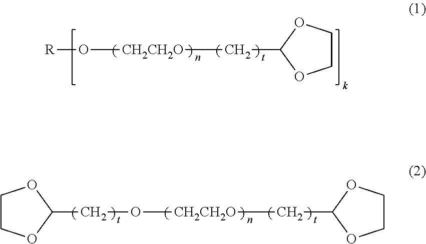

wherein, n is an integer from 20 to 1000;
t is independently an integer from 1 to 6 at each occurrence;
k is an integer from 1 to 8; when k is 1, R is selected from the group consisting of H, —CH₃, —CH₂CH₃, -TBS, -Bn, —(CH₂)ₜₚ—COOH, —(CH₂)ₜₚ—COOtBu and —(CH₂)ₜₚ—N₃; tp is an integer from 1 to 3; when k is an integer from 2 to 8, R is a multivalent branched structure, ranging from divalent to octavalent;
the preparation method comprises:
dissolving small molecule acetal derivative I-2 in an organic solvent; activating I-2 with an alkali reagent; adding polyethylene glycol derivative I-1 or I-3, each of which contains functional group X₁ and dissolving in an organic solvent; then conducting the reaction for 2 to 24 hours at 20 to 90° C. to obtain the polyethylene glycol acetal derivative represented by formula (1) or formula (2);
or dissolving polyethylene glycol derivative I-1 or I-3, each of which contains functional group X₁, in an organic solvent; activating I-1 or I-3 with an alkali reagent; adding small molecule acetal derivative I-2, which is dissolved in an organic solvent; and conducting the reaction for 2 to 24 hours at 20 to 90° C. to obtain the polyethylene glycol acetal derivative represented by formula (1) or formula (2);
wherein, X₁ is independently selected from the group consisting of H, a p-toluenesulfonyl group, and a methanesulfonyl group at each occurrence; X₂ is independently selected from the group consisting of —OH, —SH, —NH₂, —Cl, —Br, a p-toluenesulfonyl group, a methanesulfonyl group, a trifluorosulfonyl group, and a trifluoroethylsulfonyl group at each occurrence;

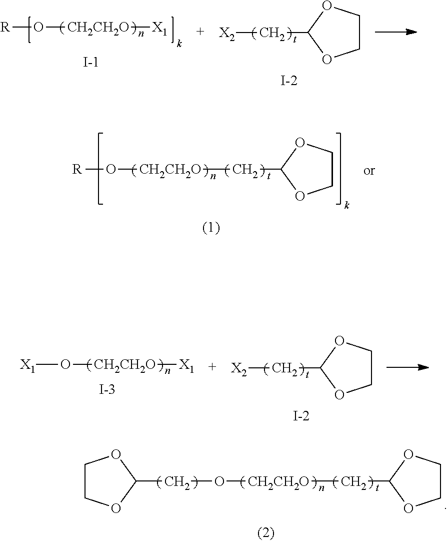

The present application also provides a method for preparing a polyethylene glycol aldehyde derivative by subjecting the acetal derivative represented by formula (1) or (2) to acid treatment, wherein the acetal derivative represented by formula (1) or (2) is prepared according to the aforementioned method.

The present application also provides a bio-related substance modified with a polyethylene glycol aldehyde derivative obtained according to the aforementioned preparation method; wherein, the bio-related substance is selected from the group consisting of a peptide, a polypeptide, a protein, a polysaccharide, a steroid, a nucleotide, an oligonucleotide, a polynucleotide, and a lipid.

### THE BENEFICIAL EFFECTS OF THE PRESENT APPLICATION

The present application provides a method for preparing polyethylene glycol acetals and derivatives thereof, wherein a series of linear and nonlinear polyethylene glycol aldehyde derivatives with single or multiple aldehyde functionalization are prepared using small molecule cyclic acetal derivatives and polyethylene glycol as starting materials in the presence of an alkali reagent. The preparation method of the present application features fewer reaction steps and eliminates the need for column chromatography for separation and purification, with no detectable amount of residual polyethylene glycol starting materials or other by-products. The method has advantages such as stability, high efficiency, environmental friendliness, cost-effectiveness, and suitability for large-scale production. The resulting products exhibit high purity and a high substitution rate of terminal functional groups, providing superior polyethylene glycol aldehyde modifiers in the field of PEGylation modification.

### BRIEF DESCRIPTION OF THE DRAWINGS

FIG. 1 is the NMR spectrum (¹HNMR) of mPEG-propionaldehyde-20k (P1-2) prepared in Example-1 of the present application.

FIG. 2 is the NMR spectrum (¹HNMR) of mPEG-butyraldehyde-20k (P2-2) prepared in Example-2 of the present application.

FIG. 3 is the NMR spectrum (¹HNMR) of PEG-dibutyraldehyde-30k (P3-2) prepared in Example-3 of the present application.

FIG. 4 is the NMR spectrum (¹HNMR) of PEG-dipropionaldehyde-3.4k (P4-2) prepared in Example-4 of the present application.

FIG. 5 is the NMR spectrum (¹HNMR) of 4-arm PEG-propionaldehyde-10k (P5-2) prepared in Example-5 of the present application.

FIG. 6 is the NMR spectrum of HO-PEG-propionaldehyde-20k (P6-2) prepared in Example-6 of the present application.

FIG. 7 is the NMR spectrum of N₃-PEG-propionaldehyde-20k (P7-2) prepared in Example-7 of the present application.

FIG. 8 is the GPC spectrum of mPEG-propionaldehyde-20k (P1-2) prepared in Example-1 of the present application.

FIG. 9 is the GPC spectrum of mPEG-butyraldehyde-20k (P2-2) prepared in Example-2 of the present application.

FIG. 10 is the GPC spectrum of PEG-dibutyraldehyde-30k (P3-2) prepared in Example-3 of the present application.

FIG. 11 is the GPC spectrum of PEG-dipropionaldehyde-3.4k (P4-2) prepared in Example-4 of the present application.

FIG. 12 is the GPC spectrum of 4-arm PEG-propionaldehyde-10k (P5-2) prepared in Example-5 of the present application.

FIG. 13 is the HPLC spectrum for testing the terminal substitution rate of mPEG-propionaldehyde-20k (P1-2) prepared in Example-1 of the present application.

FIG. 14 is the HPLC spectrum for testing the terminal substitution rate of mPEG-butyraldehyde-20k (P2-2) prepared in Example-2 of the present application.

FIG. 15 is the HPLC spectrum for testing the terminal substitution rate of PEG-dibutyraldehyde-30k (P3-2) prepared in Example-3 of the present application.

FIG. 16 is the HPLC spectrum for testing the terminal substitution rate of PEG-dipropionaldehyde-3.4k (P4-2) prepared in Example-4 of the present application.

FIG. 17 is the HPLC spectrum for testing the terminal substitution rate of 4-arm PEG-propionaldehyde-10k (P5-2) prepared in Example-5 of the present application.

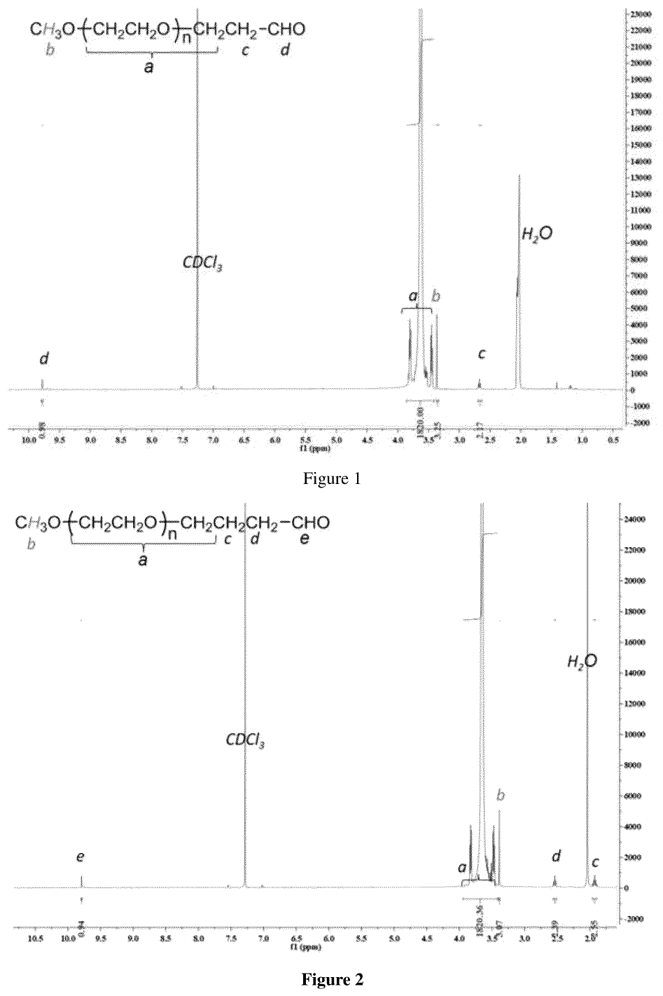

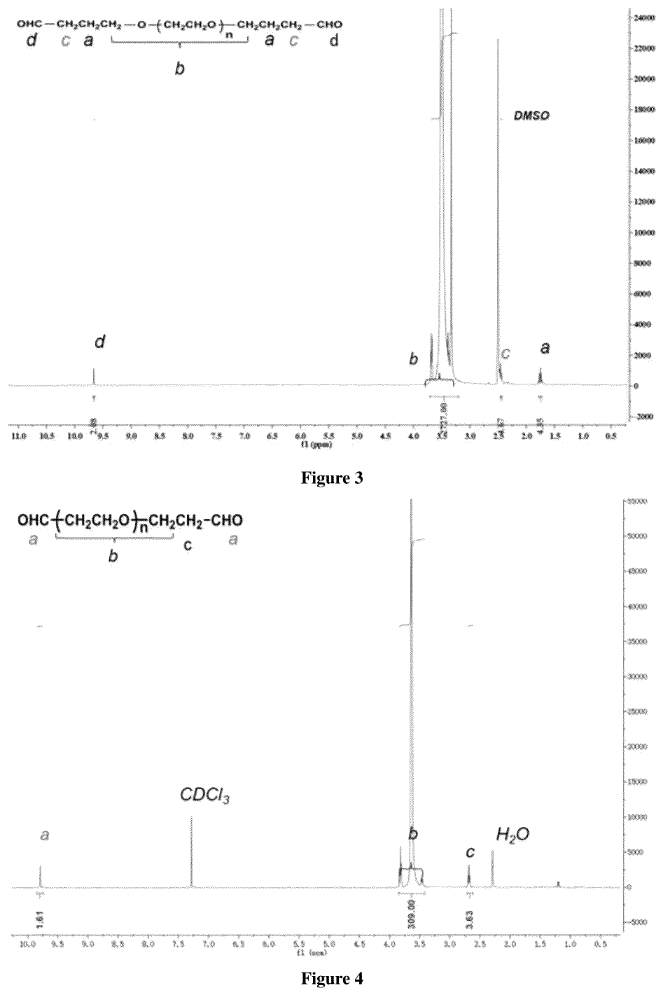

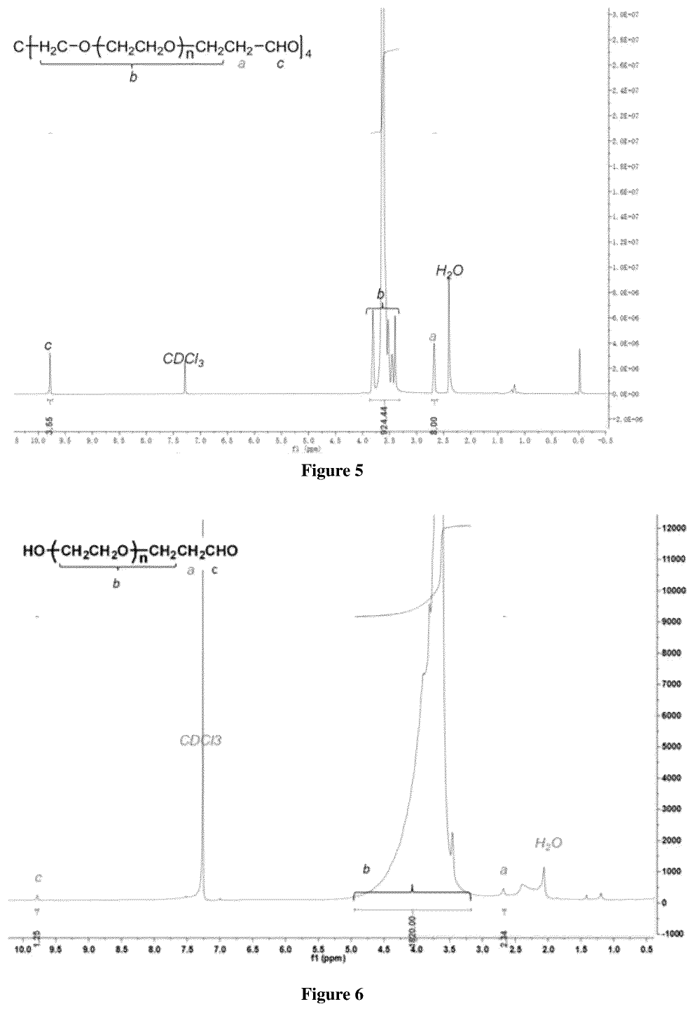

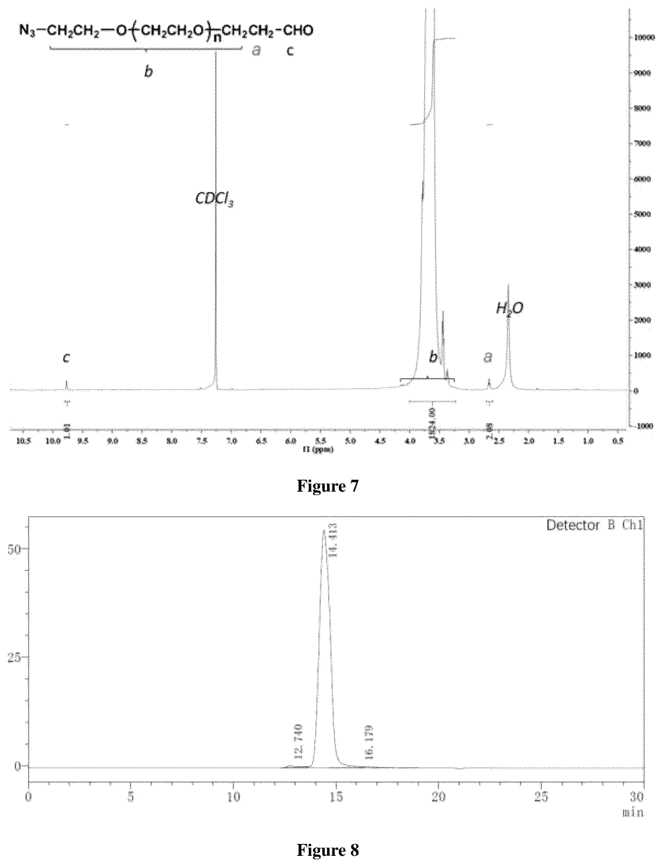

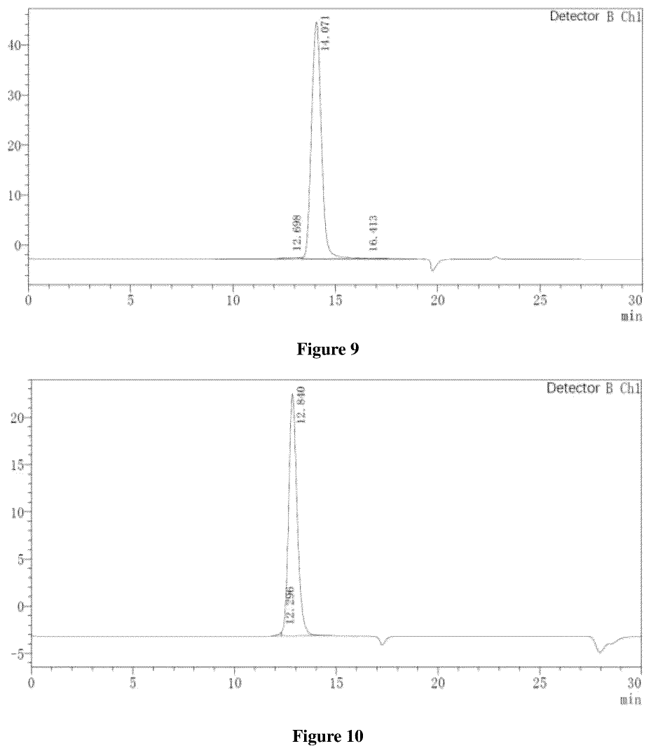

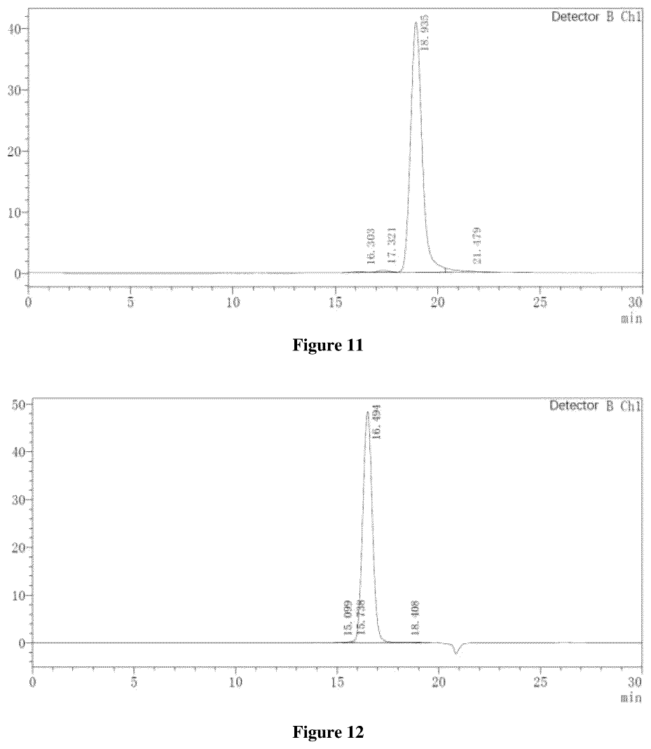

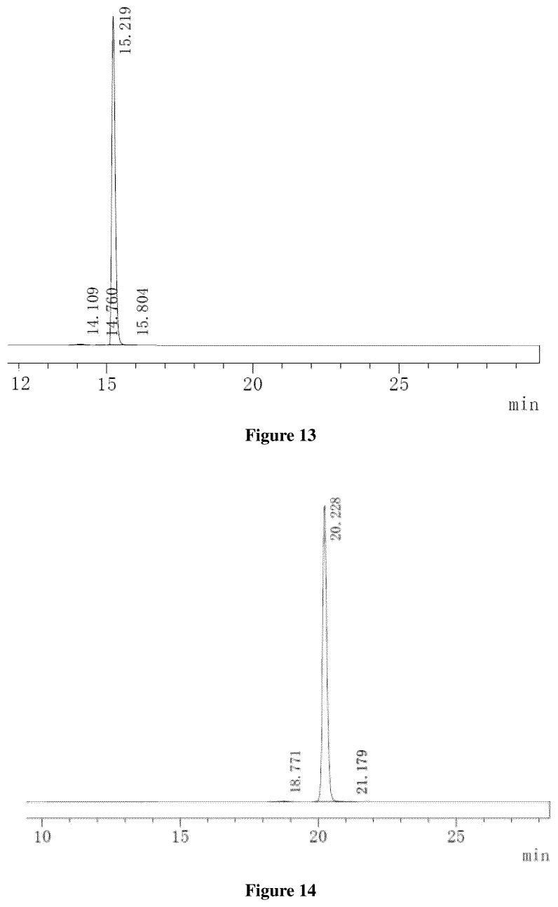

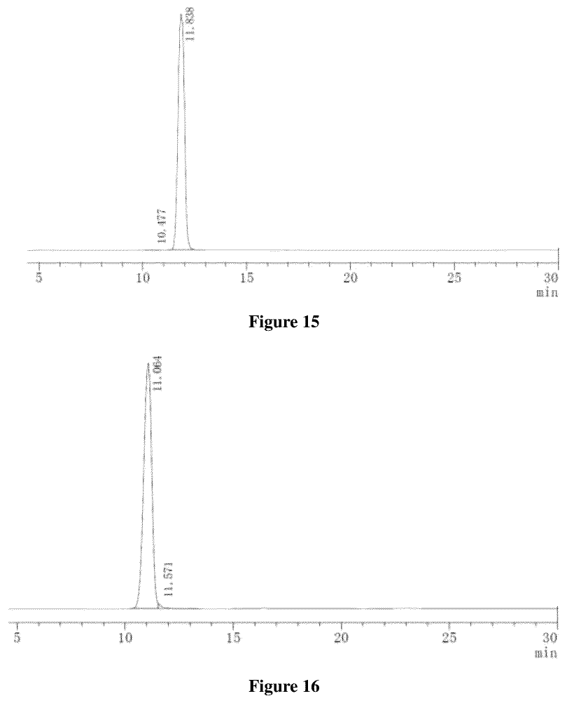

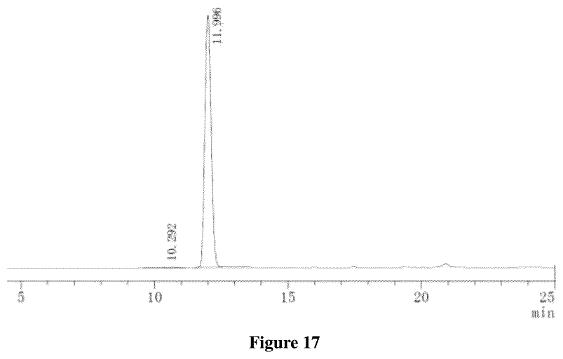

### DETAILED DESCRIPTION

### Description of Terms

The specific embodiments of the present application are described in detail. However, it should be understood that the embodiments are only given in an illustrative manner and not in a limiting manner and that various variations and modifications within the scope of the present application will be apparent to those skilled in the art.

In the present application, all technologies and scientific terms used herein have the same meaning as those commonly understood by those of ordinary skill in the art, unless otherwise described. The disclosures of all patents and other publications cited herein are incorporated by reference in their entirety. In the event of any conflict between the description and interpretation of the terms used in this application and any document incorporated by reference, the description and interpretation of the following terms shall prevail. Unless otherwise indicated, the terms have the following meanings.

In the present application, a numerical interval may be indicated by a dash (e.g., 1-6) or a tilde (e.g., 1˜6). In the present application, an integer interval represents a group consisting of all integers within the range, including both endpoints, unless otherwise specified. For example, the integer interval 1-6 represents the group consisting of 1, 2, 3, 4, 5, and 6. The numerical interval in the present application includes, but is not limited to, ranges represented by integers, non-integers, percentages, and fractions, and includes both endpoints, unless otherwise specified.

In the present application, “about” and “approximately” generally indicate a range of ±10% for numerical values.

In the present application, when a terminal group of a linking group may be easily confused with a substituent group of the linking group, “ [symbol] ” is used to mark the location where the linking group is connected to other groups. For example, in structural formulas

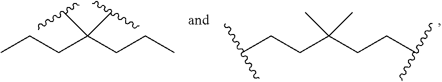

[symbol]

is used to mark the two locations where the divalent linking group is connected to other groups; the two aforementioned structural formulas represent —CH(CH₂CH₂CH₃)₂— and —CH₂CH₂CH(CH₃)₂—CH₂CH₂—, respectively.

In the present application, “molecular weight” represents the molecular mass of a compound. “Average molecular weight” represents the average molecular mass of compound components with the same general formula in a macroscopic substance; unless otherwise specified, “average molecular weight” generally refers to “number average molecular weight” (Mₙ). The number average molecular weight can be used to describe the molecular weight of either polydisperse blocks or substances, or monodisperse blocks or substances. Unless otherwise specified, the measuring unit of “molecular weight” and “average molecular weight” is Dalton (Da). The molecular weight of a polyethylene glycol chain can also be measured by “degree of polymerization” which specifically refers to the number of repeating units (oxyethylene units, EO-units) in a compound molecule. Accordingly, “average degree of polymerization” and preferably “number-average degree of polymerization” are used to characterize the average value and the number average value of the number of repeating units.

In the present application, for polydisperse cases, when a term such as “equal”, “same”, “equivalent”, or “approximately equal” (including other forms of equivalent expression) is used to describe the molecular weight or degree of polymerization of a single compound, or the number-average molecular weight or number-average degree of polymerization of a compound component in macroscopic matter, the term does not impose a strict numerical equality but indicates an approximation or approximate equality in value, unless otherwise specified; the approximation or approximate equality preferably refers to a deviation within ±10%, more preferably a deviation within ±5%, generally based on the preset value. For example, when the molecular weight of mPEG is 5 kDa, it preferably means that the molecular weight value of a single molecule with the general formula is within 4500 to 5500 Da, and the average molecular weight of the corresponding component in the preparation product is 5 kDa, that is, the product with the average molecular weight within 4500 to 5500 Da is the target product.

In the present application, unless otherwise specified, the actions of “selected from” or “preferably selected from” for any two objects are independent from each other. When there are multiple levels of selection or preferred selection, the aforementioned actions for the two objects may occur at the same or different levels. For example, “L_{A} and L_{B} are each independently selected from the group consisting of A, B, and C” includes cases where both L_{A} and L_{B} are A, or where L_{A} is A while L_{B} is B₁ (B₁ is a subset of B). Another example is that “L_{A} is preferably A (level 1 preference), more preferably A₁ to A₃ (level 2 preference), and most preferably A₁₁ to A₁₃ (level 3 preference), while L_{B} is preferably B (level 1 preference), more preferably B₁ to B₃ (level 2 preference), and most preferably B₁₁ to B₁₃ (level 3 preference)” includes cases where the preferable selections are that A is A₁ to A₃ (level 2 preference) while B is B₁₁ to B₁₃ (level 3 preference), or where both A and B are at level 3 preference.

In the present application, phrases such as “each independently”, “each independently selected from”, and “each independently, preferably selected from” may refer not only to different categories being independently defined by any option within the definitions, but may also include the phrase “at each occurrence” to indicate that the same category at different locations or occurrences is independently defined by any option within the definitions. For example, the two instances of t in formula (2) are, at each occurrence, independently an integer from 1 to 6.

In the present application, when at least two items are enumerated, the term “combination” refers to any combination of two or more of the aforementioned listed items. The quantity of each item within a combination is not limited, which may be zero, one, or greater than one; when the quantity of an item exceeds one, the specific forms of the item may be the same or different. For example, in the context of the statement “the alkali reagent is selected from the group consisting of metal alkoxides, metal hydrides, metal hydroxides, and combinations of any two or more thereof”, a combination of any two listed items may contain two specific metal alkoxides, two specific metal hydrides, or two specific metal hydroxides, which is also may be one metal alkoxide and one metal hydride, one metal alkoxide and one metal hydroxide, or one metal hydride and one metal hydroxide.

In the present application, unless otherwise specified, the term “include” and similar expressions in the specification and claims shall be interpreted in an open and inclusive manner as “include but not be limited to” or “non-restrictively include”.

In the present application, terminal substitution rate is also termed terminal activity, referring to the ratio of aldehyde groups as active functional groups present at the ends of PEG. The higher the terminal substitution rate, the higher the degree of aldehyde PEGylation, which makes the PEGylation modifier more effective.

### An Embodiment of the Present Application

A method for preparing a polyethylene glycol acetal derivative, wherein the structure of the derivative is represented by formula (1) or formula (2):

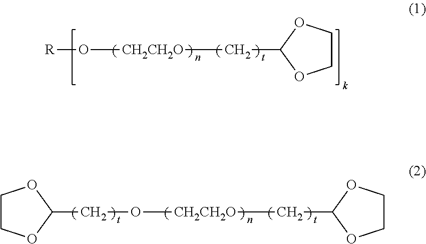

wherein, n is an integer from 20 to 1000;
t is independently an integer from 1 to 6 at each occurrence;
k is an integer from 1 to 8; when k is 1, R is selected from the group consisting of H, —CH₃, —CH₂CH₃, -TBS, -Bn, —(CH₂)ₜₚ—COOH, —(CH₂)ₜₚ—COOtBu and —(CH₂)ₜₚ—N₃; tp is an integer from 1 to 3; when k is an integer from 2 to 8, R is a multivalent branched structure, ranging from divalent to octavalent;
the preparation method comprises:
dissolving small molecule acetal derivative I-2 in an organic solvent; activating I-2 with an alkali reagent; adding polyethylene glycol derivative I-1 or I-3, each of which contains functional group X₁ and is dissolved in an organic solvent; and conducting the reaction for 2 to 24 hours at 20 to 90° C. to obtain the polyethylene glycol acetal derivative represented by formula (1) or formula (2);
or dissolving polyethylene glycol derivative I-1 or I-3, each of which contains functional group X₁, in an organic solvent; activating I-1 or I-3 with an alkali reagent; adding small molecule acetal derivative I-2, which is dissolved in an organic solvent; and conducting the reaction for 2 to 24 hours at 20 to 90° C. to obtain the polyethylene glycol acetal derivative represented by formula (1) or formula (2);
wherein, X₁ is independently selected from the group consisting of H, a p-toluenesulfonyl group, and a methanesulfonyl group at each occurrence; X₂ is independently selected from the group consisting of —OH, —SH, —NH₂, —Cl, —Br, a p-toluenesulfonyl group, a methanesulfonyl group, a trifluorosulfonyl group, and a trifluoroethylsulfonyl group at each occurrence;

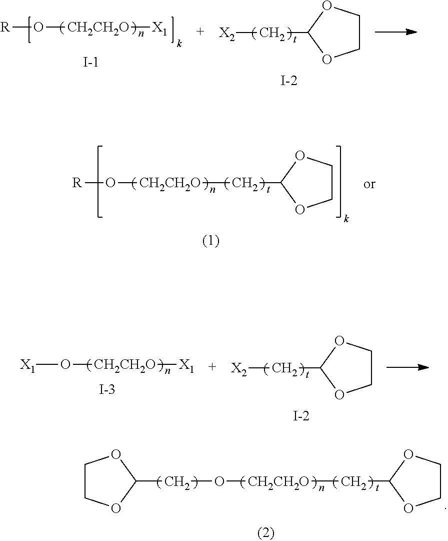

In a specific embodiment of the present application, R is preferably selected from the following cases:
Case 1: k is 1, and R is selected from the group consisting of H, —CH₃, —CH₂CH₃, -TBS, -Bn, —(CH₂)ₜₚ—COOH, —(CH₂)ₜₚ—COOtBu, —(CH₂)ₜₚNH₂, and —(CH₂)ₜₚNHCbz;
Case 2: k is 2, and R is —CH₂CH₂—;
Case 3: k is 3, and R is selected from the group consisting of

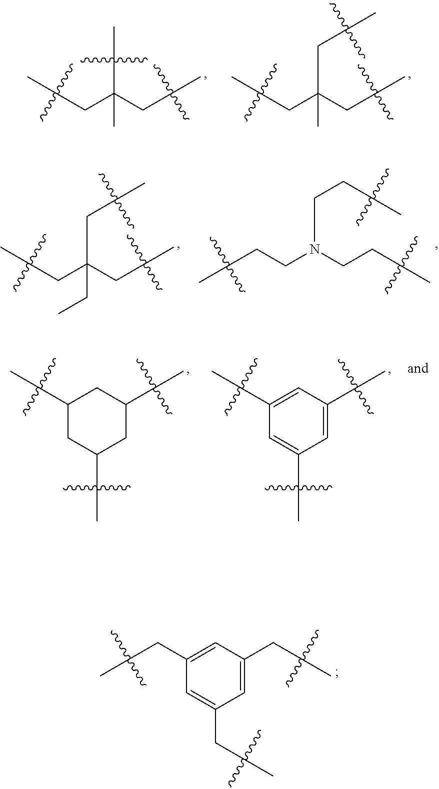

Case 4: k is 4, and R is

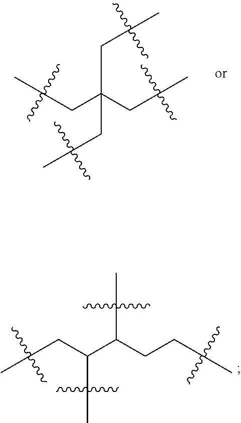 [SMILES: *CC(C*)(C*)C* ; *CCC(*)C(*)C*]

Case 5: k is 6, and R is

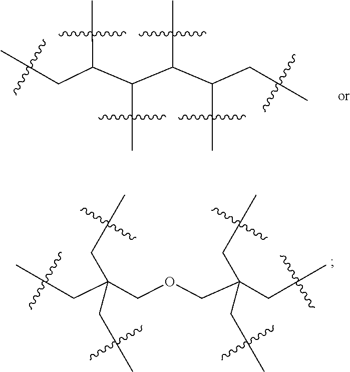 [SMILES: *CC(*)C(*)C(*)C(*)C* ; *CC(C*)(C*)COCC(C*)(C*)C*]

Case 6: k is 8, and R is

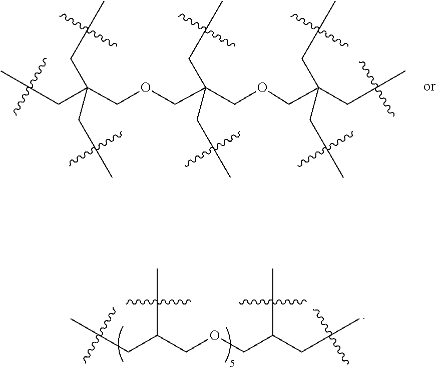

In a specific embodiment of the present application, the number average molecular weight of each polyethylene glycol chain in formula (1) or formula (2) is preferably selected from the group consisting of 900, 1000, 1500, 2000, 2500, 3000, 3350, 3400, 3500, 4000, 5000, 5500, 6000, 6500, 7000, 7500, 8000, 8500, 9000, 9500, 10000, 11000, 15000, 20000, 25000 and 30000.

In a specific embodiment of the present application, the alkali reagent in the aforementioned embodiments is preferably selected from the group consisting of metal alkoxides, metal hydrides, metal hydroxides, and combinations of any two or more thereof; the metal alkoxide is selected from the group consisting of sodium methoxide, sodium ethoxide and sodium tert-butoxide; the metal hydride is potassium hydride or sodium hydride; the metal hydroxide is sodium hydroxide or potassium hydroxide; more preferably, the alkali reagent is selected from the group consisting of sodium hydroxide, potassium hydroxide, potassium hydroxide, and sodium hydroxide.

In a specific embodiment of the present application, in formula (1) or formula (2), X₁ is preferably selected from the group consisting of H, a p-toluenesulfonate group, and a methanesulfonate group, and X₂ is preferably selected from the group consisting of —OH, —Cl, or —Br. In a specific embodiment of the present application, it is preferred that X₁ is a p-toluenesulfonyl group or a methanesulfonyl group, X₂ is —OH, and the alkali reagent is sodium hydroxide or potassium hydroxide; or it is preferred that X₁ is H, X₂ is —Cl or —Br, and the alkali reagent is sodium hydroxide or potassium hydroxide. In a specific embodiment of the present application, it is preferred that X₁ is a p-toluenesulfonate group, X₂ is —OH, and the preparation method comprises:

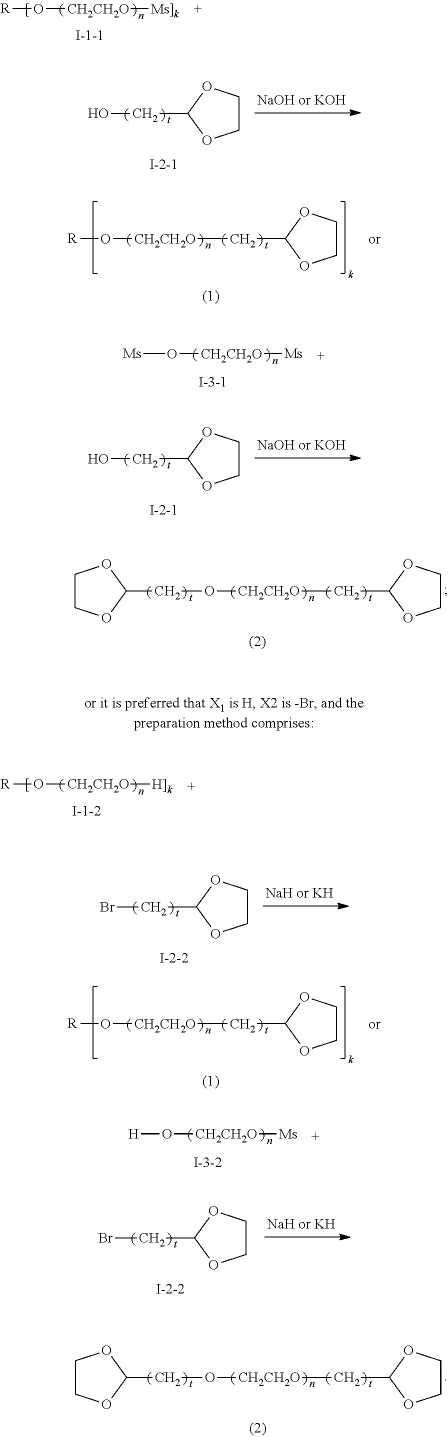

In a specific embodiment of the present application, the organic solvent is not particularly limited, provided that it is capable of dissolving the starting material and the product. The organic solvent is preferably selected from the group consisting of toluene, dichloromethane, chloroform, acetonitrile, dimethylsulfoxide, tetrahydrofuran, N,N′-dimethylformamide, N,N′-dimethylacetamide, 1,4-dioxane, and combinations of any two or more thereof, more preferably toluene or tetrahydrofuran, and most preferably anhydrous tetrahydrofuran.

In a specific embodiment of the present application, the temperature for the activation treatment with the alkali reagent in the aforementioned embodiments is preferably 20 to 60° C., more preferably 30 to 50° C., and most preferably 40° C.; the duration of the activation treatment is preferably 6 to 24 hours, more preferably 8 to 12 hours; the molar ratio of small molecule acetal derivative I-2 to the alkali reagent is preferably from 1:1 to 2:1; more preferably from 1:1 to 1.5:1, and most preferably 1:1, 1.25:1, or 1.2:1.

In a specific embodiment of the present application, the molar ratio of I-1 to small molecule acetal derivative I-2 is preferably from 1:10 to 1:160; when k is 1, the molar ratio of I-1 to I-2 is preferably from 1:10 to 1:30; the molar ratio of polyethylene glycol derivative I-3 containing functional group X₁ to small molecule acetal derivative I-2 is preferably from 1:20 to 1:100.

In a specific embodiment of the present application, the reaction is preferably conducted under an inert gas, and the inert gas is selected from the group consisting of nitrogen, helium, argon, and any combination thereof.

In a specific embodiment of the present application, the starting material I-1 or I-3 for the reaction is added dropwise to I-2; the purification process is conducted with precipitation or recrystallization; the precipitating reagent is an ether reagent, selected from an ethyl ether or a methyl tert-butyl ether; the recrystallization reagent is selected from the group consisting of isopropanol, n-hexane, and any combination thereof; more preferably, the recrystallization reagent is a mixture of isopropanol and n-hexane, and the volume ratio of isopropanol to n-hexane is from 2:1 to 5:1.

In a specific embodiment of the present application, the structure of I-1 is preferably represented by

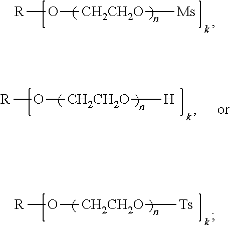

wherein, the definition of R is the same as that described in formula (1).

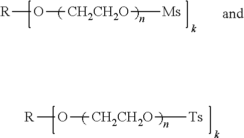

can be obtained through the methanesulfonylation or p-toluenesulfonylation of their corresponding alcohols; for example, H₃CO [symbol] CH₂CH₂O [symbol] Ms or H₃CO [symbol] CH₂CH₂O [symbol] Ts can be obtained through the methanesulfonylation or p-toluenesulfonylation of H₃CO [symbol] CH₂CH₂O [symbol] H, while

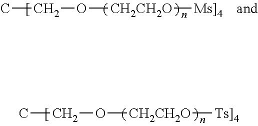

can be obtained through the methanesulfonylation or p-toluenesulfonylation of

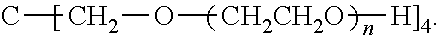

In a specific embodiment of the present application, the structure of I-1 is preferably selected from the group consisting of

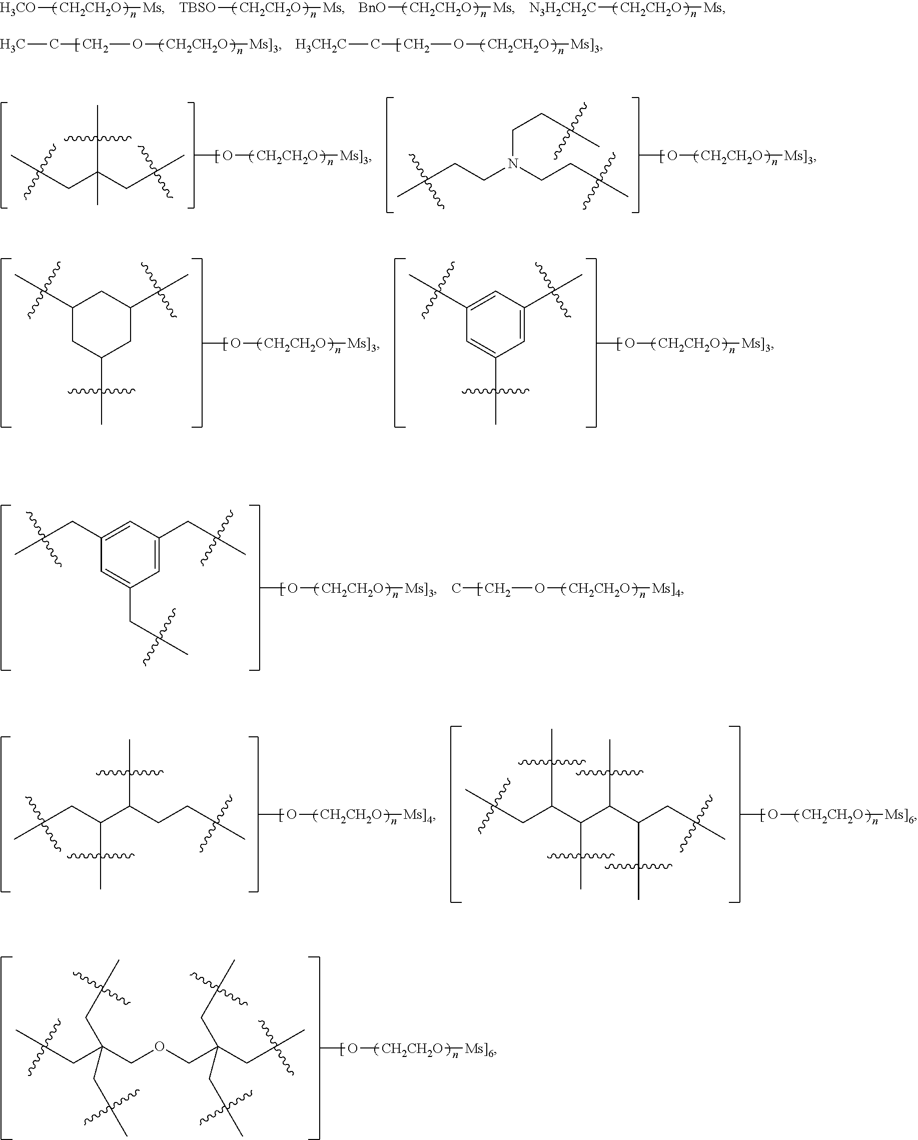

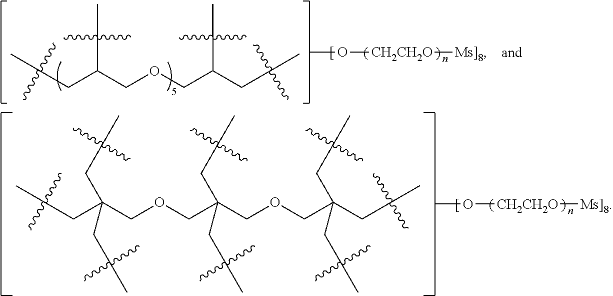

In a specific embodiment of the present application, the structure of I-3 is preferably

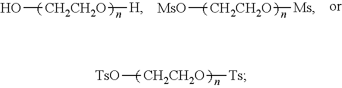

similarly,

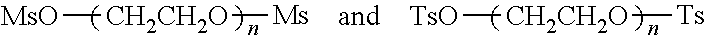

can be obtained through the methanesulfonylation or p-toluenesulfonylation of

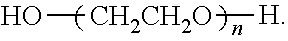

In a specific embodiment of the present application, the structure of I-2 is preferably selected from the group consisting of

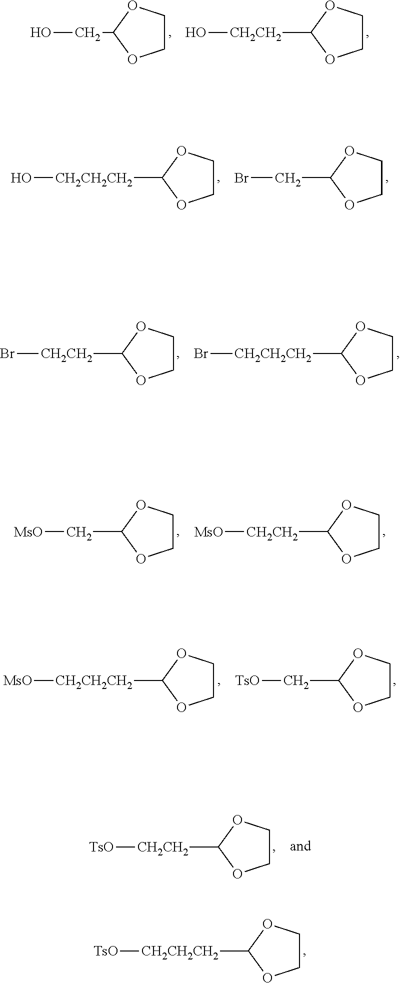

### Another Embodiment of the Present Application

A method for preparing a polyethylene glycol aldehyde derivative, wherein the method comprises subjecting a polyethylene glycol acetal derivative, which is prepared by any of the aforementioned methods, to acid treatment. In the present application, almost all of the polyethylene glycol acetal derivatives are converted to the corresponding polyethylene glycol aldehyde derivatives, and the ¹HNMR spectra of polyethylene glycol aldehyde derivatives do not exhibit signals of —CH<protons of cyclic acetal groups (chemical shift approximately 4.80 to 5.00) and contain characteristic peaks for aldehyde groups (chemical shift approximately 9.70 to 9.90).

In one specific embodiment of the present application, the acid used in the preparation of the polyethylene glycol aldehyde derivative is selected from the group consisting of hydrochloric acid, trifluoroacetic acid, formic acid, acetic acid and mixtures thereof.

In one specific embodiment of the present application, the structures of the polyethylene glycol aldehydes prepared according to the described method include but are not limited to the following structures:

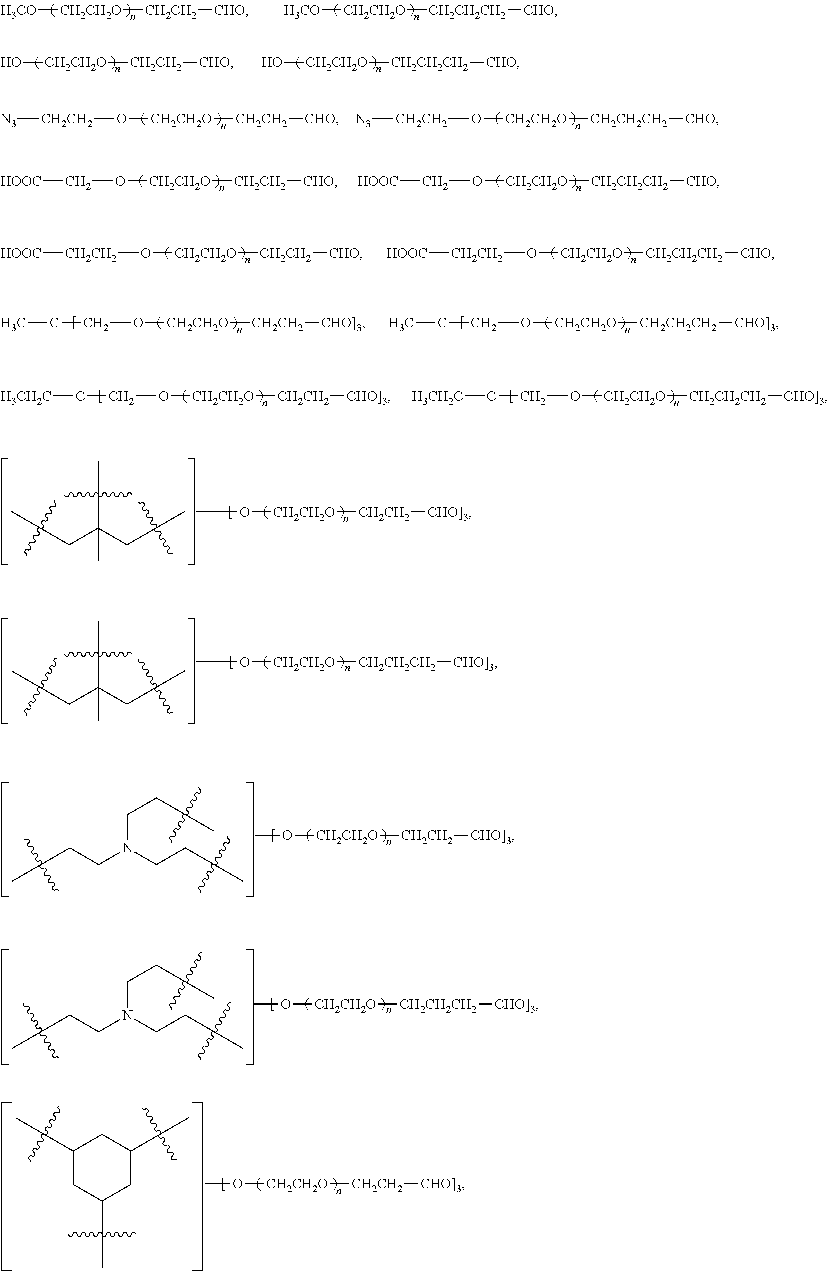

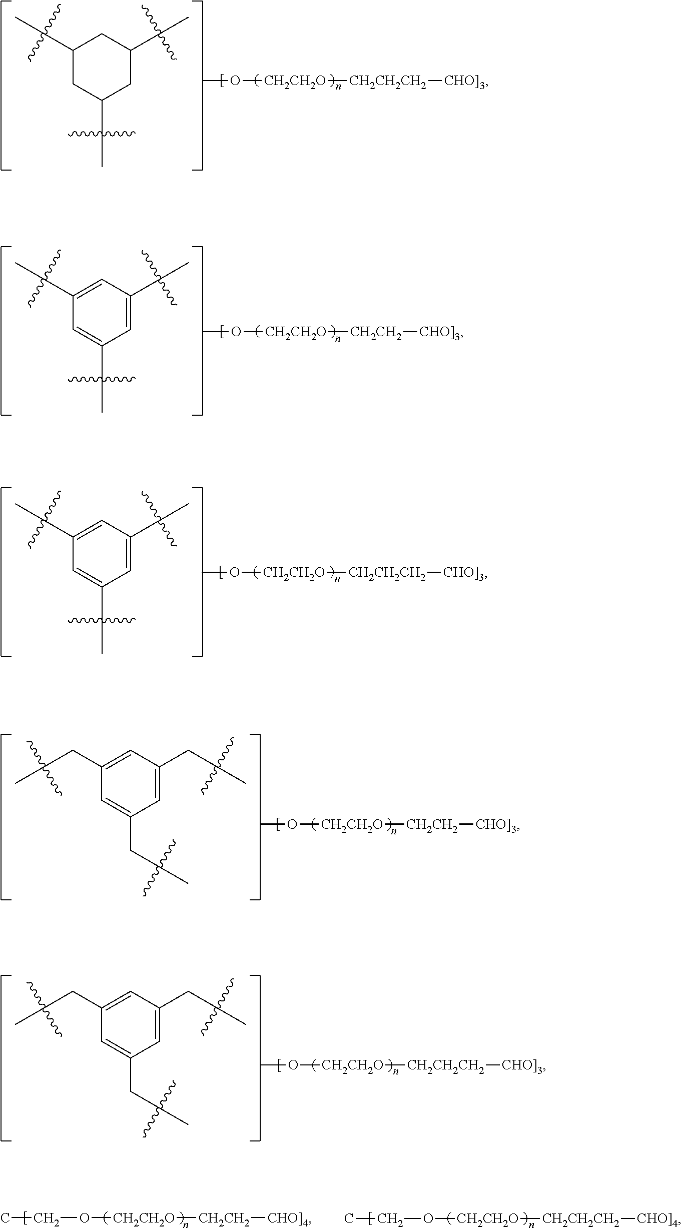

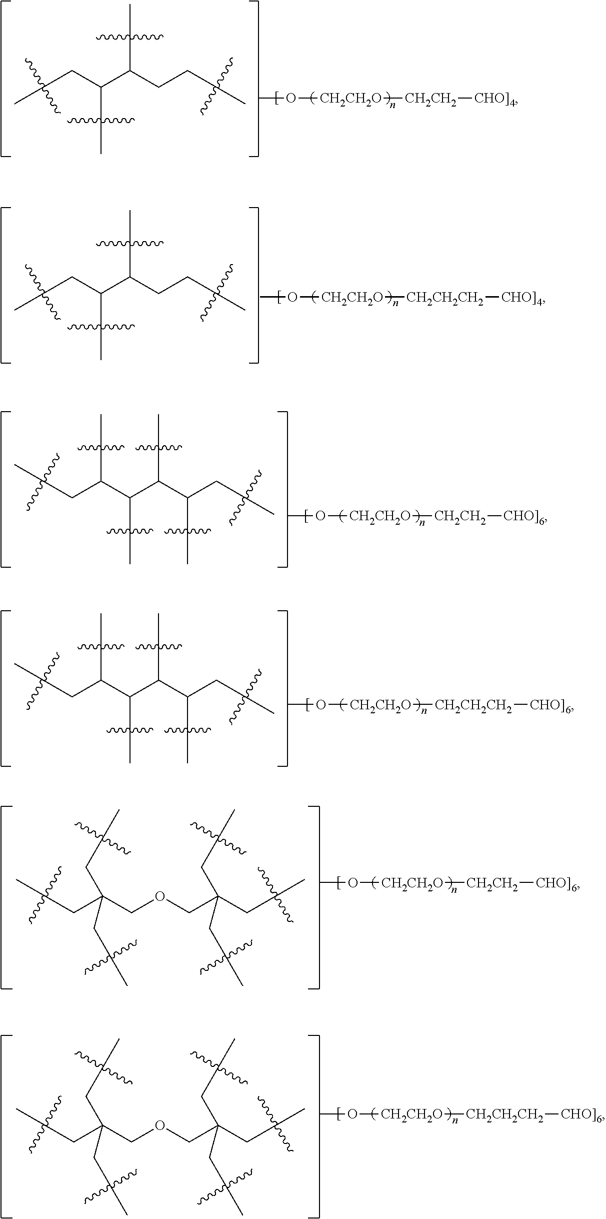

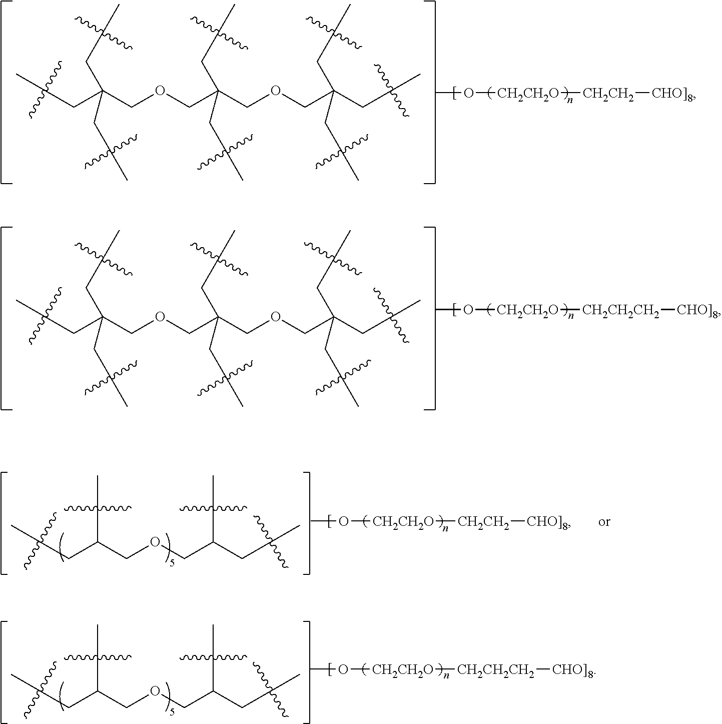

In the present application, the starting materials used in each preparation method can be commercially purchased or synthesized independently.

### Another Embodiment of the Present Application

A bio-related substance modified with a polyethylene glycol aldehyde derivative prepared according to the aforementioned preparation method; wherein, the bio-related substance is selected from the group consisting of a peptide, a polypeptide, a protein, a polysaccharide, a steroid, a nucleotide, an oligonucleotide, a polynucleotide, and a lipid; more preferably, the bio-related substance contains an active amino group, or, after modification, contains at least one active amino group capable of coupling with the aldehyde group of the polyethylene glycol aldehyde derivative.

The following is a further description with specific examples of the preparation methods of polyethylene glycol acetal derivatives and polyethylene glycol aldehyde derivatives. The specific examples are provided for illustrative purposes only and do not limit the scope of protection of the present application. The intermediates and end products prepared in the present application can be purified by purification methods including but not limited to extraction, recrystallization, adsorption treatment, precipitation, counter precipitation, etc. The structure and molecular weight of the end product may be characterized by characterization methods including but not limited to NMR, electrophoresis, UV-visible spectrophotometry, FTIR, AFM, GPC, HPLC, MALDI-TOF, circular dichroism, etc. In the present application, the structures are preferably characterized by ¹H-NMR, the number average molecular weight (Mn) and polydispersity index (PDI) are preferably determined by gel permeation chromatography (GPC), and the terminal substitution rates are preferably analyzed by high-performance liquid chromatography (HPLC).

### Example-1: Preparation of mPEG-propionaldehyde

### Example-1.1: Preparation of mPEG-propionaldehyde-20k (P1-2)

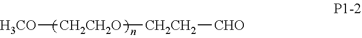

The preparation process is as follows:

Step a: under nitrogen protection, S1-1 (0.89 g, 7.5 mmol) and anhydrous THF (50 mL) were added to a dry and clean 250 mL round-bottom flask, and NaOH (0.25 g, 6.25 mmol) was added in an ice bath and stirred until dissolved. The reaction was stirred at 40° C. for 8 hours. After the reaction was completed, the reaction solution was cooled to room temperature. mPEG-OMs-20k (5.00 g, 0.25 mmol, Mn=20 kDa), dissolved in anhydrous THF (50 mL), was added dropwise to the aforementioned reaction solution. After the addition was completed, the reaction was stirred at 40° C. for 8 hours. After the reaction was completed, the reaction solution was cooled to room temperature, and then added with 10% NaCl solution, washed three times with ethyl acetate, and extracted twice with dichloromethane. The organic phases were combined, dried with anhydrous sodium sulfate, filtered, concentrated, and recrystallized with anhydrous isopropanol/n-hexane (v/v=3/1) to obtain mPEG-propylal-20k (P1-1, 4.66 g, yield: 92.7%). ¹HNMR (CDCl₃, 400 MHz) δ (ppm): 1.87-1.99 (—CH₂CH₂CH<, 2H), 3.36-3.39 (—OCH₃, 3H), 3.40-3.80 (—OCH₂CH₂O—, PEG-H; —OCH₂CH₂CH<, 2H), 3.82-3.98 (>CHOCH₂CH₂O<, 4H), 4.93-4.99 (>CHCH₂—, 1H).

Step b: P1-1 prepared in Step a was added to a dry and clean 500 mL round-bottom flask, followed by adding 1 N HCl solution, and then the reaction was stirred in an ice bath for 8 hours. After the reaction was completed, the pH of the solution was adjusted to 6.4±0.2 with sodium bicarbonate, and then the solution was extracted twice with dichloromethane. The organic phases were combined, dried with anhydrous magnesium sulfate, filtered, and recrystallized with the mixture of anhydrous isopropanol and n-hexane to obtain mPEG-propionaldehyde-20k (P1-2, yield: 98%). The characteristic peak of the acetal group disappeared and the characteristic peak of the aldehyde group 9.76-9.80 (—CHO, 1H) appeared in the spectrum of ¹HNMR (CDCl₃). The terminal substitution rate of the aldehyde group in P1-2 was determined to be 99% by HPLC. The GPC analysis showed that Mn was 20.1 kDa and PDI was 1.03.

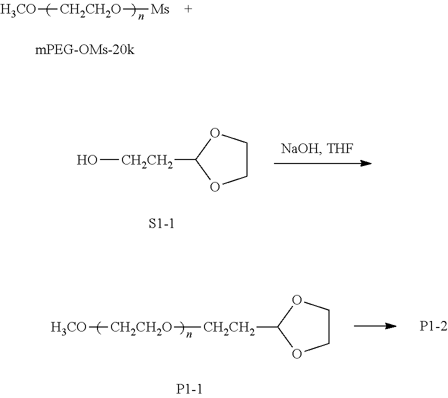

### Example-1.2: Preparation of mPEG-propionaldehyde-2k (P1-4)

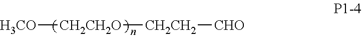

The preparation process is as follows:

Step a: under nitrogen protection, S1-1 (23.63 g, 200.00 mmol) and anhydrous THF (200 mL) were added to a dry and clean 500 mL round-bottom flask, and NaOH (8.00 g, 200.00 mmol) was added to the solution in an ice bath and stirred until dissolved. The reaction was stirred at 40° C. for 8 hours. After the reaction was completed, the reaction solution was cooled to room temperature. mPEG-OMs-20k (20.00 g, 10.00 mmol, Mn=2 kDa), dissolved in anhydrous THF (200 mL), was added dropwise to the aforementioned reaction solution. After the addition was completed, the reaction was stirred at 40° C. for 8 hours. After the reaction was completed, the reaction solution was cooled to room temperature, and then added with 10% NaCl solution, washed three times with ethyl acetate, and extracted twice with dichloromethane. The organic phases were combined, dried with anhydrous sodium sulfate, filtered, concentrated, and recrystallized with anhydrous isopropanol/n-hexane (v/v=3/1) to obtain mPEG-propylal-2k (P1-3, 19.53 g, yield: 93%). ¹HNMR (CDCl₃, 400 MHz) δ (ppm): 1.86-1.99 (—CH₂CH₂CH<, 2H), 3.35-3.38 (—OCH₃, 3H), 3.40-3.80 (—OCH₂CH₂O—, PEG-H; —OCH₂CH₂CH₂CH<, 2H), 3.83-3.98 (>CHOCH₂CH₂O<, 4H), 4.93-4.99 (>CHCH₂—, 1H).

Step b: P1-3 prepared in Step a was added to a dry and clean 1 L round-bottom flask, followed by adding 1 N HCl solution, and the reaction was stirred in an ice bath for 8 hours. After the reaction was completed, the pH of the solution was adjusted to 6.4±0.2 with sodium bicarbonate, and then the solution was extracted twice with dichloromethane. The organic phases were combined, dried with anhydrous magnesium sulfate, filtered, and recrystallized with the mixture of anhydrous isopropanol and n-hexane to obtain mPEG-propionaldehyde-2k (P1-4, yield: 98%). The characteristic peak of the acetal group disappeared and the characteristic peak of the aldehyde group 9.76-9.80 (—CHO, 1H) appeared in the spectrum of ¹HNMR (CDCl₃, 400 MHz). The terminal substitution rate of the aldehyde group in P1-4 was determined to be 99% by HPLC. The GPC analysis showed that Mn was 2.1 kDa and PDI was 1.03.

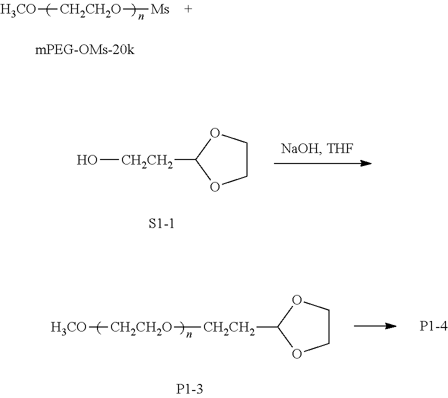

### Example-2: Preparation of mPEG-butyraldehyde-20k (P2-2)

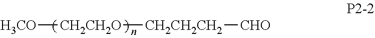

The preparation process is as follows:

Step a: under nitrogen protection, S2-1 (0.99 g, 7.50 mmol) and anhydrous THF (100 mL) were added to a dry and clean 250 mL round-bottom flask. Then, NaOH (0.20 g, 5.00 mmol) was added in an ice bath and stirred until dissolved. The reaction was stirred at 40° C. for 8 hours. After the reaction was completed, the reaction solution was cooled to room temperature. mPEG-OMs-20k (5.00 g, 0.25 mmol, Mn=20 kDa), dissolved in anhydrous THF (50 mL), was added dropwise to the aforementioned reaction solution. After the addition was completed, the reaction was stirred at 55° C. for 8 hours. After completion of the reaction, the reaction solution was cooled to room temperature, and then added with 10% NaCl solution, washed three times with ethyl acetate, and extracted twice with dichloromethane. The organic phases were combined, dried with anhydrous sodium sulfate, filtered, concentrated, and recrystallized with anhydrous isopropanol/n-hexane (v/v=3/1) to obtain mPEG-butyral-20k (P2-1, 4.68 g, yield: 93.1%). ¹HNMR (CDCl₃, 400 MHz) δ (ppm): 1.73-1.82 (—CH₂CH₂CH₂CH<, 4H), 3.32-3.37 (—OCH₃, 3H), 3.38-3.80 (—OCH₂CH₂O—, PEG-H; —OCH₂CH₂CH₂CH<, 2H), 3.84-4.09 (>CHOCH₂CH₂O<, 4H), 4.82-4.90 (>CHCH₂—, 1H).

Step b: P2-1 prepared in Step a was added to a dry and clean 500 mL round-bottom flask, followed by adding 1 N HCl solution, and the reaction was stirred in an ice bath for 8 hours. After the reaction was completed, the pH of the solution was adjusted to 6.4±0.2 with sodium bicarbonate, and then the solution was extracted twice with dichloromethane. The organic phases were combined, dried with anhydrous magnesium sulfate, filtered, and recrystallized with the mixture of anhydrous isopropanol and n-hexane to obtain mPEG-butyraldehyde-20k (P2-2, yield: 97%). The characteristic peak of the acetal group disappeared and the characteristic peak of the aldehyde group 9.75-9.78 (—CHO, 1H) appeared in the spectrum of ¹HNMR (CDCl₃, 400 MHz). The terminal substitution rate of the aldehyde group in P2-2 was determined to be 99% by HPLC. The GPC analysis showed that Mn was 20.1 kDa and PDI was 1.03.

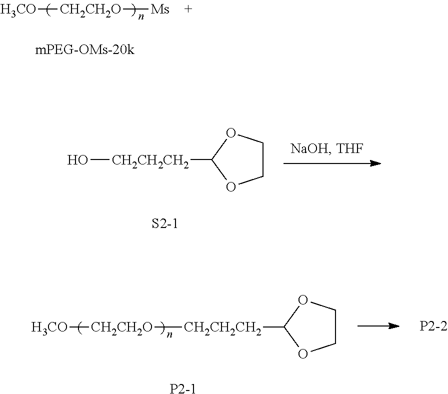

### Example-3: Preparation of PEG-30k-dibutyraldehyde (P3-2)

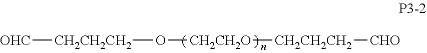

The preparation process is as follows:

Step a: S2-1 (4.36 g, 33.00 mmol) and anhydrous THF (50 mL) were added to a dry and clean 1 L round-bottom flask. Then, NaOH (4.26 g, 106.00 mmol) was added in an ice bath and stirred until dissolved. The reaction was stirred at 40° C. for 8 hours. After the reaction was completed, the reaction solution was cooled to room temperature. MSO-PEG-OMs-30k (9.90 g, 0.33 mmol, Mn=30 kDa), dissolved in anhydrous THF (100 mL), was added dropwise to the aforementioned reaction solution. After the addition was completed, the reaction was stirred at 55° C. for 8 hours. After completion of the reaction, the reaction solution was cooled to room temperature, and then added with 10% NaCl solution, washed three times with ethyl acetate, and extracted twice with dichloromethane. The organic phases were combined, dried with anhydrous sodium sulfate, filtered, concentrated, and recrystallized with anhydrous isopropanol/n-hexane (v/v=3/1) to obtain PEG-dibutyral-30k (P3-1, 9.31 g, yield: 93.8%). ¹HNMR (d₆-DMSO, 400 MHz) δ (ppm): 1.50-1.65 (—OCH₂CH₂CH₂CH<, 8H), 3.35-3.65 (—OCH₂CH₂O—, PEG-H; —OCH₂CH₂OCH₂—, 4H), 3.70-3.90 (>CHOCH₂CH₂O<, 8H), 4.75-4.85 (>CHCH₂—, 2H).

Step b: P3-1 prepared in Step a was added to a dry and clean 2 L round-bottom flask, followed by adding 1 N HCl solution, and the reaction was stirred in an ice bath for 8 hours. After completion of the reaction, the pH of the solution was adjusted to 6.4±0.2 with sodium bicarbonate, and then the solution was extracted twice with dichloromethane. The organic phases were combined, dried with anhydrous magnesium sulfate, filtered, and recrystallized with the mixture of anhydrous isopropanol and n-hexane to obtain PEG-dibutyraldehyde-30k (P3-2, yield: 97%). The characteristic peak of the acetal group disappeared and the characteristic peak of the aldehyde group 9.79-9.83 (—CHO, 2H) appeared in the spectrum of ¹HNMR (d₆-DMSO, 400 MHz). The terminal substitution rate of the aldehyde group in P3-2 was determined to be 99% by HPLC. The GPC analysis showed that Mn was 30.2 kDa and PDI was 1.04.

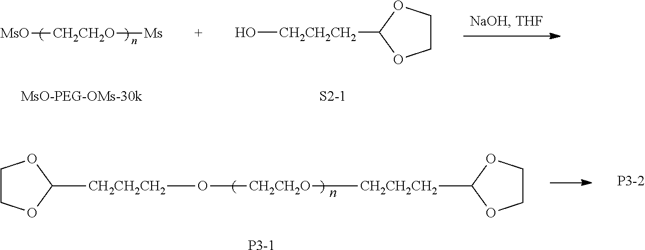

### Example-4: Preparation of PEG-dipropionaldehyde-3.4k (P4-2)

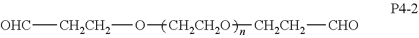

The preparation process is as follows:

Step a: under nitrogen protection, S1-1 (93.56 g, 792.00 mmol) and anhydrous THF (300 mL) were added to a dry and clean 1 L round-bottom flask. Then, NaOH (21.12 g, 528.00 mmol) was added in an ice bath and stirred until dissolved. The reaction was stirred at 40° C. for 8 hours. After completion of the reaction, the reaction solution was cooled to room temperature. MsO-PEG-OMs-3.4k (29.92 g, 8.80 mmol, Mn=3.4 kDa), dissolved in anhydrous THF (300 mL), was added dropwise to the aforementioned reaction solution. After the addition was completed, the reaction was stirred at 40° C. for 8 hours. After the reaction was completed, the reaction solution was cooled to room temperature, and then added with 10% NaCl solution, washed three times with ethyl acetate, and extracted twice with dichloromethane. The organic phases were combined, dried with anhydrous sodium sulfate, filtered, concentrated, and recrystallized with anhydrous isopropanol/n-hexane (v/v=3/1) to obtain PEG-dipropylal-3.4k (P4-1, 28.14 g, yield: 92.2%). ¹HNMR (CDCl₃, 400 MHz) δ (ppm): 1.86-1.99 (—CH₂CH₂CH<, 4H), 3.38-3.80 (—OCH₂CH₂O—, PEG-H; —OCH₂CH₂CH<, 4H), 3.83-3.98 (>CHOCH₂CH₂O<, 8H), 4.93-4.99 (>CHCH₂—, 2H).

Step b: P4-1 prepared in Step a was added to a dry and clean 2 L round-bottom flask, followed by adding 1 N HCl solution, and the reaction was stirred in an ice bath for 8 hours. After the reaction was completed, the pH of the solution was adjusted to 6.4±0.2 with sodium bicarbonate, and then the solution was extracted twice with dichloromethane. The organic phases were combined, dried with anhydrous magnesium sulfate, filtered, and recrystallized with the mixture of anhydrous isopropanol and n-hexane to obtain PEG-dipropionaldehyde-3.4k (P4-2, yield: 97%). The characteristic peak of the acetal group disappeared and the characteristic peak of the aldehyde group 9.74-9.85 (—CHO, 2H) appeared in the spectrum of ¹HNMR (CDCl₃, 400 MHz). The terminal substitution rate of the aldehyde group in P4-2 was determined to be 99% by HPLC. The GPC analysis showed that Mn was 3.6 kDa and PDI was 1.03.

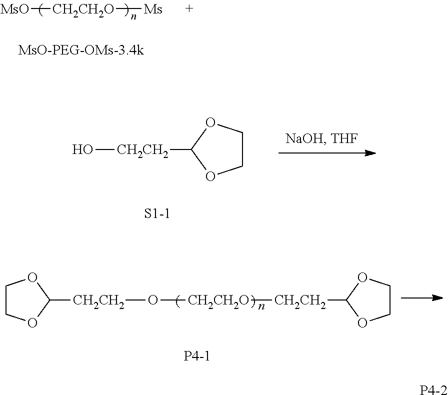

### Example-5: Preparation of 4-arm PEG-propionaldehyde

### Example-5.1: Preparation of 4-arm PEG-propionaldehyde 4-arm-PEG-pALD-10k

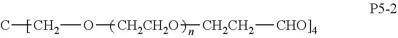

The preparation process is as follows:

Step a: under nitrogen protection, 4-arm-PEG-OH (25.00 g, 2.50 mmol, Mn=10 kDa) was added to a dry and clean 2 L round-bottom flask, and water was removed through azeotropic distillation with toluene. NaH (16.00 g, 400.00 mmol), dissolved in anhydrous THF (1 L), was added to the aforementioned 4-arm-PEG-OH solution. The reaction temperature was raised to 85° C. and the reaction was refluxed for 8 hours. After the reaction was completed, the reaction solution was cooled to room temperature, and then added with NaI (1.50 g, 10.00 mmol) and S5-1 (72.41 g, 400.00 mmol). The reaction temperature was raised to 85° C. and the reaction was refluxed for 8 hours. After the reaction was completed, the mixture was placed in an ice bath, and an aqueous solution of ammonium chloride was slowly added dropwise to quench the reaction. Then, THF was removed by concentration, followed by adding 10% NaCl solution. The mixture was washed three times with ethyl acetate and then extracted twice with dichloromethane. The organic phases were combined, dried with anhydrous sodium sulfate, filtered, concentrated, and recrystallized with anhydrous isopropanol/n-hexane (v/v=3/1) to obtain 4-arm-PEG-propylal-10k (P5-1, 23.59 g, yield: 90.7%). ¹HNMR (CDCl₃, 400 MHz) δ (ppm): 1.85-1.97 (—CH₂CH₂CH<, 8H), 3.40-3.80 (—OCH₂CH₂O—, PEG-H; —OCH₂CH₂CH<, 8H; —CCH₂—, 8H), 3.83-3.98 (>CHOCH₂CH₂O<, 16H), 4.91-4.97 (>CHCH₂—, 4H).

Step b: P5-1 prepared in Step a was added to a dry and clean 2 L round-bottom flask, followed by adding 1 N HCl solution, and the reaction was stirred in an ice bath for 8 hours. After the reaction was completed, the pH of the solution was adjusted to 6.4±0.2 with sodium bicarbonate, and then the solution was extracted twice with dichloromethane. The organic phases were combined, dried with anhydrous magnesium sulfate, filtered, and recrystallized with the mixture of anhydrous isopropanol and n-hexane to obtain 4-arm-PEG-propionaldehyde-10k (P5-2, yield: 97%). The characteristic peak of the acetal group disappeared and the characteristic peak of the aldehyde group 9.76-9.82 (—CHO, 4H) appeared in the spectrum of ¹HNMR (CDCl₃, 400 MHz). The terminal substitution rate of aldehyde group in P5-2 was determined to be 97% by HPLC. The GPC analysis showed that Mn was 10.4 kDa and PDI was 1.04.

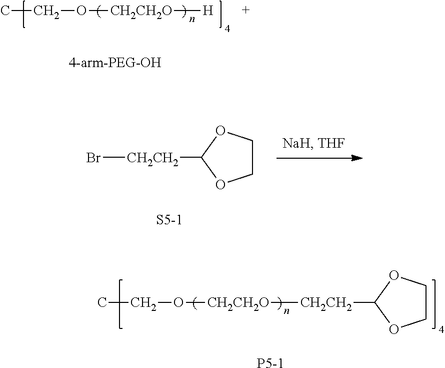

### Example-5.2: Preparation of 4-arm PEG-propionaldehyde 4-arm-PEG-pALD-20k

Following the procedures in Example-5.1, the starting material 4-arm-PEG-OH-10k was replaced with 4-arm-PEG-OH-20k to obtain 4-arm-PEG-propylal-20k (P5-3) and 4-arm-PEG-propanal-20k (P5-4). The structures were also verified by ¹HNMR, GPC, and HPLC.

### Example-6: Preparation of hydroxy-PEG-propionaldehyde-20k (P6-2)

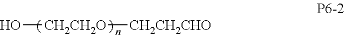

The preparation process is as follows:

Step a: under nitrogen protection, S1-1 (0.89 g, 7.50 mmol) and anhydrous THF (50 mL) were added to a dry and clean 250 mL round-bottom flask. Then, NaOH (0.20 g, 5.00 mmol) was added in an ice bath and stirred until dissolved. The reaction was stirred at 40° C. for 8 hours. After the reaction was completed, the reaction solution was cooled to room temperature. TBSO-PEG-OMs-20k (5.00 g, 0.25 mmol), dissolved in anhydrous THF (50 mL), was added dropwise to the aforementioned reaction solution. After the addition was complete, the reaction was stirred at 40° C. for 8 hours. After the reaction was completed, the reaction solution was cooled to room temperature, and then added with 10% NaCl solution, washed three times with ethyl acetate, and extracted twice with dichloromethane. The organic phases were combined, dried with anhydrous sodium sulfate, filtered, concentrated, and recrystallized with anhydrous isopropanol/n-hexane (v/v=3/1) to obtain TBSO-PEG-propylal-20k (P6-1, 4.65 g, yield: 93.0%). ¹HNMR (CDCl₃, 400 MHz) δ (ppm): 0.21 (—Si(CH₃)₂—, 6H), 0.98 (—SiC(CH₃)₃, 9H), 1.75-1.84 (—CH₂CH₂CH<, 2H), 3.35-3.72 (—OCH₂CH₂O—, PEG-H; —OCH₂CH₂CH₂CH<, 2H), 3.71-3.90 (>CHOCH₂CH₂O<, 4H), 4.80-4.90 (>CHCH₂—, 1H).

Step b: P6-1 prepared in Step a was added to a dry and clean 500 mL round-bottom flask and dissolved with a mixed solution of tetrahydrofuran/acetic acid. TBAF was added in an ice bath. The reaction was conducted at room temperature for 8 hours. After extraction and concentration, 1 N HCl solution was added to the reaction solution and the reaction was stirred in an ice bath for 8 hours. After completion of the reaction, the pH of the solution was adjusted to 6.4±0.2 with sodium bicarbonate, and then the solution was extracted twice with dichloromethane. The organic phases were combined, dried with anhydrous magnesium sulfate, filtered, and recrystallized with the mixture of anhydrous isopropanol and n-hexane to obtain hydroxy-PEG-propionaldehyde-20k (P6-2, yield: 95%). The characteristic peaks of the acetal and TBS groups disappeared and the characteristic peak of the aldehyde group 9.73-9.80 (—CHO, 1H) appeared in the spectrum of ¹HNMR (CDCl₃, 400 MHz). The terminal substitution rate of the aldehyde group in P6-2 was determined to be 97% by HPLC. The GPC analysis showed that Mn was 20.1 kDa and PDI was 1.04.

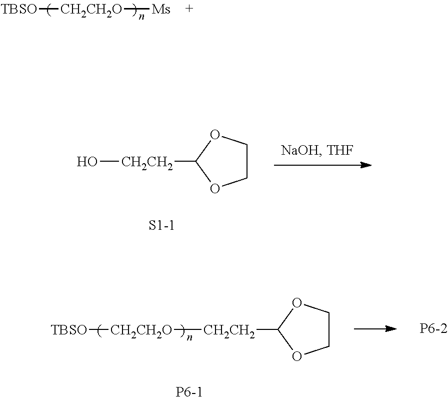

### Example-7: Preparation of N₃-PEG-propionaldehyde-20k (P7-2)

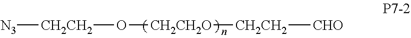

The preparation process is as follows:

Step a: under nitrogen protection, S1-1 (0.89 g, 7.50 mmol) and anhydrous THF (50 mL) were added to a dry and clean 250 mL round-bottom flask. Then, NaOH (0.20 g, 5.00 mmol) was added under in ice bath and stirred until dissolved, and the reaction was stirred at 40° C. for 8 hours. After the reaction was completed, the reaction solution was cooled to room temperature. N₃-PEG-OMs-20k (5.00 g, 0.25 mmol), dissolved in anhydrous THF (50 mL), was added dropwise to the aforementioned reaction solution. After the addition was completed, the reaction was stirred at 40° C. for 8 hours. After completion of the reaction, the reaction solution was cooled to room temperature, and then added with 10% NaCl solution, washed three times with ethyl acetate, and extracted twice with dichloromethane. The organic phases were combined, dried with anhydrous sodium sulfate, filtered, concentrated, and recrystallized with anhydrous isopropanol/n-hexane (v/v=3/1) to obtain N₃-PEG-propylal-20k (P7-1, 4.65 g, yield: 93.0%). ¹HNMR (CDCl₃, 400 MHz) δ (ppm): 1.75-1.84 (—CH₂CH₂CH<, 2H), 3.35-3.72 (—OCH₂CH₂O—, PEG-H; —OCH₂CH₂CH₂CH<, 2H; —OCH₂CH₂N₃, 4H), 3.71-3.90 (>CHOCH₂CH₂O<, 4H), 4.80-4.90 (>CHCH₂—, 1H).

Step b: P7-1 prepared in Step a and 1 N HCl solution were successively added to a dry and clean 500 mL round-bottom flask, and the mixture was stirred in an ice bath for 8 hours. After the reaction was completed, the pH of the solution was adjusted to 6.4±0.2 with sodium bicarbonate, and then the solution was extracted twice with dichloromethane. The organic phases were combined, dried with anhydrous magnesium sulfate, filtered, and recrystallized with the mixture of anhydrous isopropanol and n-hexane to obtain N₃-PEG-propionaldehyde-20k (P7-2, yield: 95%). The characteristic peak of the acetal group disappeared and the characteristic peak of the aldehyde group 9.73-9.80 (—CHO, 1H) appeared in the spectrum of ¹HNMR (CDCl₃, 400 MHz). The terminal substitution rate of the aldehyde group in P7-2 was determined to be 99% by HPLC. The GPC analysis showed that Mn was 20.1 kDa and PDI was 1.03.

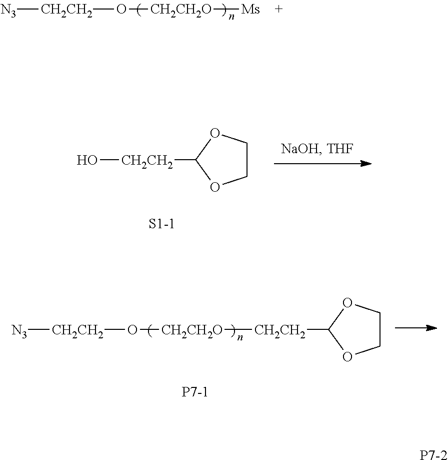

### Example-8: Preparation Method 2 of mPEG-propionaldehyde-20k (P1-2)

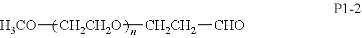

The preparation process is as follows:

Step a: under nitrogen protection, mPEG-OH (5.00 g, 0.25 mmol, Mn=20 kDa) was added to a dry and clean 2 L round-bottom flask, and water was removed through azeotropic distillation with toluene. NaH (0.20 g, 5.00 mmol), dissolved in anhydrous THF (50 mL), was added to the aforementioned mPEG-OH solution in an ice bath. The temperature was raised to 85° C. and the reaction was refluxed for 8 hours. After the reaction was completed, S5-1 (1.36 g, 7.50 mmol) was added to the reaction solution at room temperature. The temperature was raised to 85° C. and the reaction was refluxed for 8 hours. After completion of the reaction, the mixture was placed in an ice bath, and an aqueous solution of ammonium chloride was slowly added dropwise to quench the reaction. The solution was concentrated to remove THF and then added with 10% NaCl solution, washed three times with ethyl acetate, and extracted twice with dichloromethane. The organic phases were combined, dried with anhydrous sodium sulfate, filtered, concentrated, and recrystallized with anhydrous isopropanol/n-hexane (v/v=3/1) to obtain mPEG-propylal-20k (P1-1, 2.30 g, yield: 88.5%). The structure was also characterized by NMR.

Step b: P1-1 prepared in Step a and 1 N HCl solution was successively added to a dry and clean 2 L round-bottom flask, and the reaction was stirred in an ice bath for 8 hours. After completion of the reaction, the pH of the solution was adjusted to 6.4±0.2 with sodium bicarbonate, and then the solution was extracted twice with dichloromethane. The organic phases were combined, dried with anhydrous magnesium sulfate, filtered, and recrystallized with the mixture of anhydrous isopropanol and n-hexane to obtain mPEG-propionaldehyde-20k (P1-2, yield: 97%). The characteristic peak of the acetal group disappeared and the characteristic peak of the aldehyde group 9.76-9.82 (—CHO, 1H) appeared in the spectrum of ¹HNMR (CDCl₃, 400 MHz). The terminal substitution rate of the aldehyde group in P1-2 was determined to be 95% by HPLC.

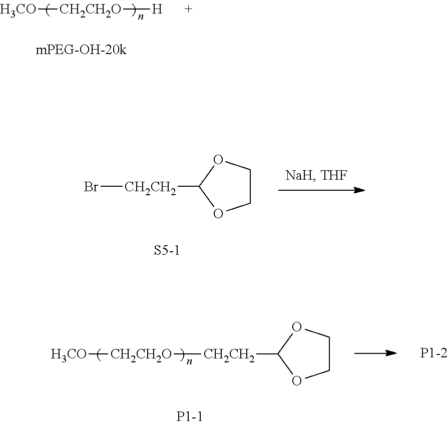

### Example-9: Preparation Method 3 of mPEG-propionaldehyde-20k (P1-2)

The alkali reagent NaH in Example-8 was replaced with NaOH, while the other steps remained the same as those in Example-8, resulting in a final yield of P1-2 of 84%. The terminal substitution rate of the aldehyde group in P1-2 was determined to be 99% by HPLC.

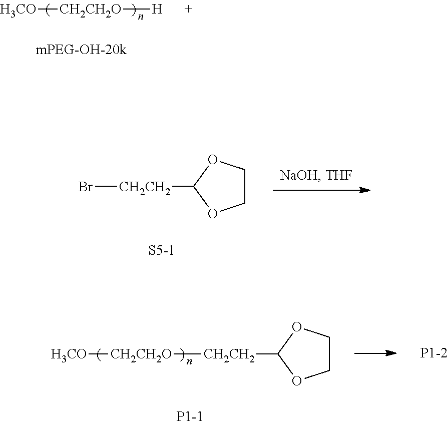

### Example-10: Preparation Method 4 of mPEG-propionaldehyde-20k (P1-2)

The preparation process is as follows:

The small molecule cyclic acetal starting material S5-1 in Example-8 was replaced with the small molecule linear acetal starting material S10-1, while the other steps remained the same as those in Example-8, resulting in a final yield of P1-2 of about 83%. The terminal substitution rate of the aldehyde group in P1-2 was determined to be 85% by HPLC.

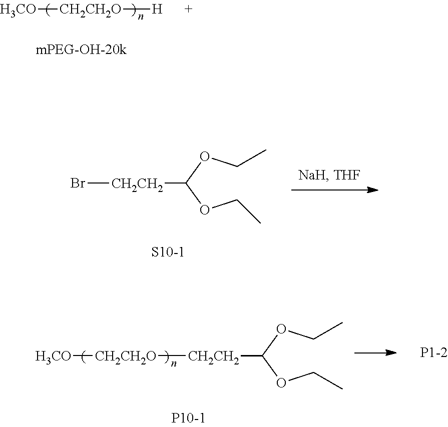

### Example-11: Preparation Method 5 of mPEG-propionaldehyde-20k (P1-2)

The preparation process is as follows:

The small molecule cyclic acetal starting material S1-1 in Example-1.1 was replaced with the small molecule linear acetal starting material S11-1, while the other steps remained the same as those in Example-1.1, resulting in a final yield of P1-2 of about 87%. The terminal substitution rate of the aldehyde group in P1-2 was determined to be 87% by HPLC.

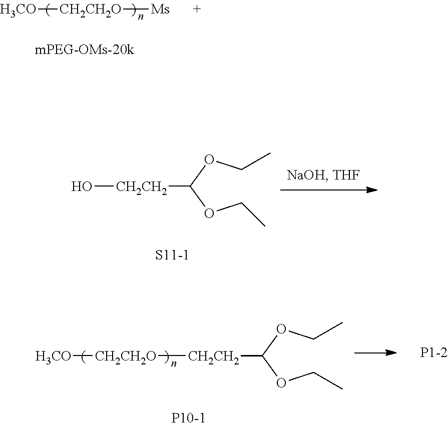

### Example-12: Preparation Method 6 of mPEG-propionaldehyde-20k (P1-2)

The starting material mPEG-OMs-20k in Example-1.1 was not added dropwise but directly added as a solid, while the other steps remained the same as those in Example-1.1, resulting in a final yield of P1-2 of about 82%. The terminal substitution rate of the aldehyde group in P1-2 was determined to be 91% by HPLC.

### Example-13: Preparation Method 7 of mPEG-propionaldehyde-20k (P1-2)

The recrystallization procedure in Step a of Example-1.1 was replaced with pulping and precipitation with an ether reagent (t-butyl methyl ether). Specifically, the crude product was dissolved in a small amount of DCM, and the solution was added dropwise to the ether reagent (where the volume of the ether reagent was 20 times that of DCM). All other steps remained the same as those in Example-1.1, resulting in a final yield of P1-2 of about 87%. The terminal substitution rate of the aldehyde group in P1-2 was determined to be 89% by HPLC.

### Example-14: Preparation Method 8 of mPEG-propionaldehyde-20k (P1-2)

The recrystallization procedure in Step a of Example-1.1 was replaced with recrystallization with isopropanol, while the other steps remained the same as those in Example-1.1, resulting in a final yield of P1-2 of about 89%. The terminal substitution rate of the aldehyde group in P1-2 was determined to be 93% by HPLC.

The above Example-1.1 and Example-8 to 14 are different preparation methods of mPEG-propionaldehyde-20k (P1-2), while Example-9 to 14 are comparative methods. The comparisons of each method are shown in Tables 1 and 2 below:

TABLE 1

| | | | | Terminal |
|---|---|---|---|---|
| | | Metallic | | Substitution Rate |
| Example | Acetal Reagent | Alkali | Final Yield (%) | (%) |
| 1.1 | 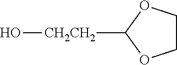 | NaOH | 91 | 99 |
| 8 | 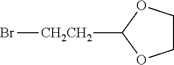 | NaH | 86 | 95 |
| 9 | 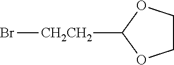 | NaOH | 84 | 38 |
| 10 | 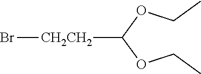 | NaH | 83 | 85 |
| 11 | 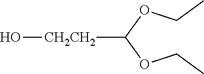 | NaOH | 87 | 87 |

TABLE 2

| | | Feeding | | |
|---|---|---|---|---|
| | | Method of | Final | Terminal |
| | Precipitation or | PEG Starting | Yield | Substitution |
| Example | Recrystallization | Material | (%) | Rate (%) |
| 1.1 | recrystallization with | dropwise | 91 | 99 |
| | anhydrous isopropanol/ | | | |
| | n-hexane (v/v = 3/1) | | | |
| 12 | recrystallization with | non- | 82 | 91 |
| | anhydrous isopropanol/ | dropwise | | |
| | n-hexane (v/v = 3/1) | | | |
| 13 | precipitation with t-butyl | dropwise | 87 | 89 |
| | methyl ether | | | |
| 14 | recrystallization with | dropwise | 89 | 93 |
| | anhydrous isopropanol | | | |

As shown in Tables 1 and 2 above, there are differences in yield and terminal functional group substitution rate among the various preparation methods, with the difference in terminal substitution rate being more significant. Those skilled in the art know that the terminal substitution rate is more important to the quality of macromolecular products, and the products with a higher terminal substitution rate generally exhibit greater purity.

According to Table 1, in the preparation of polyethylene glycol aldehyde derivatives, the types of small molecule acetal reagents and alkali reagents have a significant impact on the yield and quality of the product. Firstly, a comparison of Examples-1.1, 8, 9 and Examples-10, 11 shows that the use of small molecule cyclic acetal reagents results in a higher yield and a higher terminal substitution rate compared to small molecule linear acetal reagents, which is because the cyclic acetal group is more stable than the linear acetal group and therefore fewer side reactions are expected during the preparation process. Secondly, a comparison of Examples-1.1, 8, and 9 shows that different small molecule acetal reagents correspond to different preferable metallic alkali reagents, wherein NaOH in Example-1.1 is used for dehydrogenation activation of small molecule acetal, NaH in Example-8 and NaOH in Example-9 are used for dehydrogenation activation of the hydrogen in mPEG-OH. The dehydrogenation activation of mPEG-OH is more effective with NaH than with NaOH, resulting in a terminal aldehyde group substitution rate of 95% for the product obtained with NaH, compared to only 38% for the product obtained with NaOH.

According to Table 2, the method of adding starting materials and the post-treatment process have a significant impact on the yield and quality of the products. Firstly, comparing Example-1.1 with Example-12, it can be seen that when the polyethylene glycol starting material is added to the metallic alkali-activated small molecule acetal derivatives, the yield from dropwise addition is higher than that of direct pouring, and the terminal functional group substitution rate of the product is also higher. This may be due to the fact that the starting material added in the dropwise manner can immediately react with the activated other reactant upon addition, so that the reaction is more complete and more time-efficient. Secondly, a comparison of Examples-1.1, 13 and 14 shows that the yield from recrystallization is higher and the product quality is better than that from the precipitation method in post-treatment. Additionally, the selection of the recrystallization reagent also affects the yield and quality of the product, and the use of the mixture of isopropanol and n-hexane for the recrystallization gives a higher yield and higher substitution rate of terminal functional groups in the product.

In summary, compared with the comparative Examples 9 to 14, the product obtained by the preparation method of the present application (Example-1.1) has a higher yield, greater purity, and better quality (including a higher substitution rate of the terminal aldehyde group).

1-16. (canceled)

17. A method for preparing a polyethylene glycol acetal derivative, wherein the structure of the derivative is represented by formula (1) or formula (2): 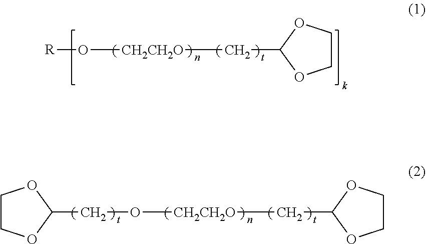
wherein, n is an integer from 20 to 1000;
t is independently an integer from 1 to 6 at each occurrence;
k is an integer from 1 to 8; when k is 1, R is selected from the group consisting of H, —CH₃, —CH₂CH₃, -TBS, -Bn, —(CH₂)ₜₚ—COOH, —(CH₂)ₜₚ—COOtBu, —(CH₂)ₜₚ—N₃; tp is an integer from 1 to 3; when k is an integer from 2 to 8, R is a multivalent branched structure, ranging from divalent to octavalent;
the preparation method comprises:
dissolving small molecule acetal derivative I-2 in an organic solvent; activating I-2 with an alkali reagent; adding polyethylene glycol derivative I-1 or I-3, each of which contains functional group X₁ and dissolving in an organic solvent; and then conducting the reaction for 2 to 24 hours at 20 to 90° C. to obtain the polyethylene glycol acetal derivative represented by formula (1) or formula (2); the alkali reagent is sodium hydroxide or potassium hydroxide;
wherein, X₁ is independently a p-toluenesulfonyl group or a methanesulfonyl group at each occurrence; X₂ is —OH;

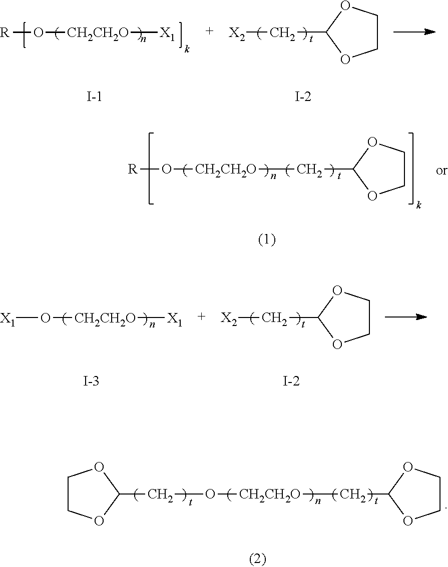

18. The method for preparing polyethylene glycol acetal derivative according to claim 17, wherein R is selected from the group consisting of H, —CH₃, —CH₂CH₃, -Bn, —(CH₂)ₜₚ—COOH, —(CH₂)ₜₚ—COOtBu, —(CH₂)ₜₚNH₂, —(CH₂)ₜₚNHCbz, —CH₂CH₂—,

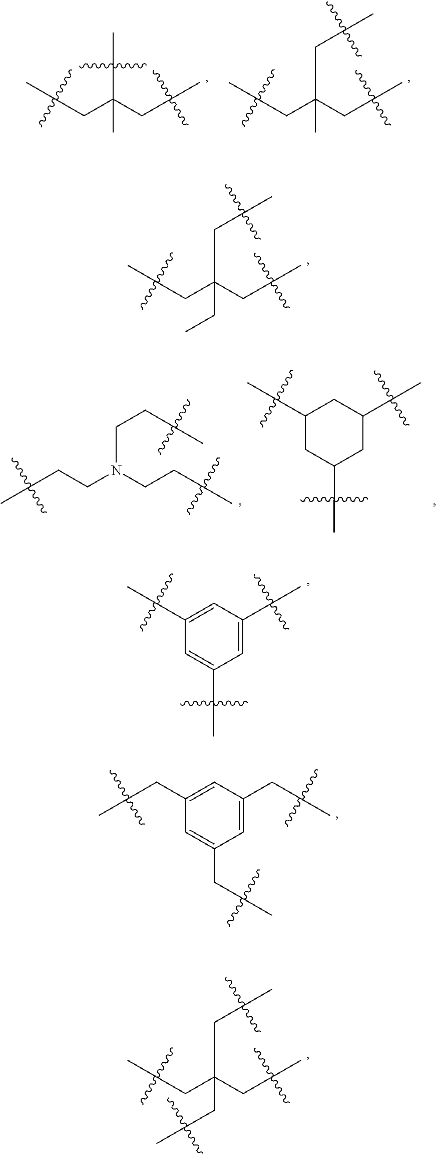

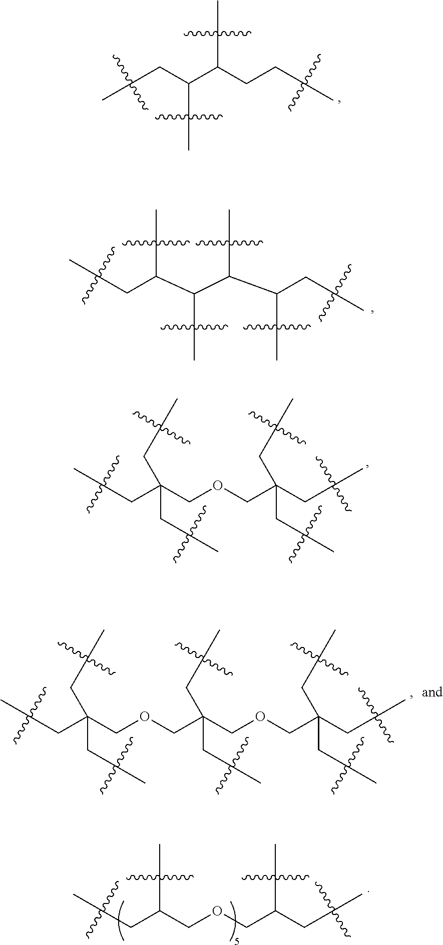

19. The method for preparing polyethylene glycol acetal derivative according to claim 17, wherein the number average molecular weight of each polyethylene glycol chain is selected from the group consisting of 900, 1000, 1500, 2000, 2500, 3000, 3350, 3400, 3500, 4000, 5000, 5500, 6000, 6500, 7000, 7500, 8000, 8500, 9000, 9500, 10000, 11000, 15000, 20000, 25000, and 30000.

20. The method for preparing polyethylene glycol acetal derivative according to claim 17, wherein when X₁ is a methanesulfonyl group, X₂ is —OH, and the preparation method comprises:

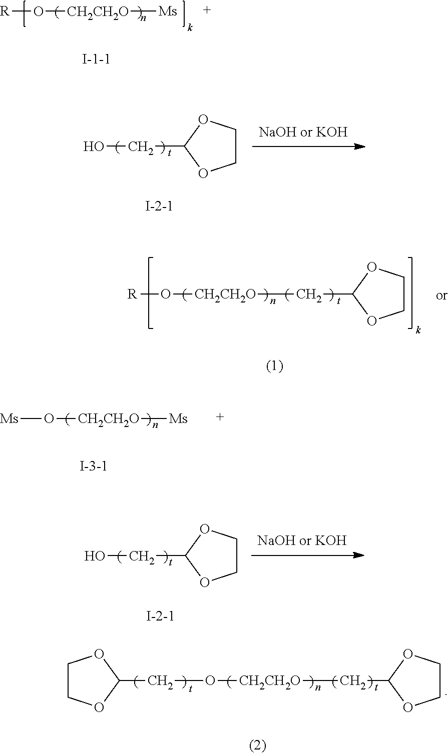

21. The method for preparing polyethylene glycol acetal derivative according to claim 17, wherein the organic solvent is selected from the group consisting of toluene, dichloromethane, chloroform, acetonitrile, dimethylsulfoxide, tetrahydrofuran, N,N′-dimethylformamide, N,N′-dimethylacetamide, 1,4-dioxane, and combinations of any two or more thereof, and more specifically is anhydrous tetrahydrofuran.

22. The method for preparing polyethylene glycol acetal derivative according to claim 17, wherein the temperature for the activation treatment is 20 to 60° C.; the duration of the activation treatment is 6 to 24 hours; the molar ratio of small molecule acetal derivative I-2 to the alkali reagent is from 1:1 to 2:1.

23. The method for preparing polyethylene glycol acetal derivative according to claim 17, wherein the temperature is 40° C.; the duration of the activation treatment is 8 to 12 hours; the molar ratio of small molecule acetal derivative I-2 to the alkali reagent is 1:1, 1.25:1, or 1.2:1.

24. The method for preparing polyethylene glycol acetal derivative according to claim 17, wherein the molar ratio of polyethylene glycol derivative I-1 containing a functional group X₁ to small molecule acetal derivative I-2 is from 1:10 to 1:160; when k is 1, the molar ratio of I-1 to I-2 is from 1:10 to 1:30; the molar ratio of polyethylene glycol derivative I-3 containing functional group X₁ to small molecule acetal derivative I-2 is from 1:20 to 1:100.

25. The method for preparing polyethylene glycol acetal derivative according to claim 17, wherein the reaction is conducted under an inert gas, and the inert gas is selected from the group consisting of nitrogen, helium, argon, and any combination thereof.

26. The method for preparing polyethylene glycol acetal derivative according to claim 17, wherein the starting material I-1 or I-3 for the reaction is added dropwise to I-2; the purification process is conducted with precipitation or recrystallization; the precipitating reagent is an ether reagent, selected from an ethyl ether or a methyl tert-butyl ether; the recrystallization reagent is selected from the group consisting of isopropanol, n-hexane, and any combination thereof.

27. The method for preparing polyethylene glycol acetal derivative according to claim 26, the recrystallization reagent is a mixture of isopropanol and n-hexane, and the volume ratio of isopropanol to n-hexane is from 2:1 to 5:1.

28. A method for preparing polyethylene glycol aldehyde derivative, wherein the method comprises subjecting a polyethylene glycol acetal derivative, which is prepared by the method of claim 17, to acid treatment.

29. The method for preparing polyethylene glycol aldehyde derivative according to claim 28, wherein the acid is selected from the group consisting of hydrochloric acid, trifluoroacetic acid, formic acid, acetic acid, and mixtures thereof.

30. The method for preparing polyethylene glycol aldehyde derivative according to claim 28, wherein the structures of the polyethylene glycolal derivatives comprises:

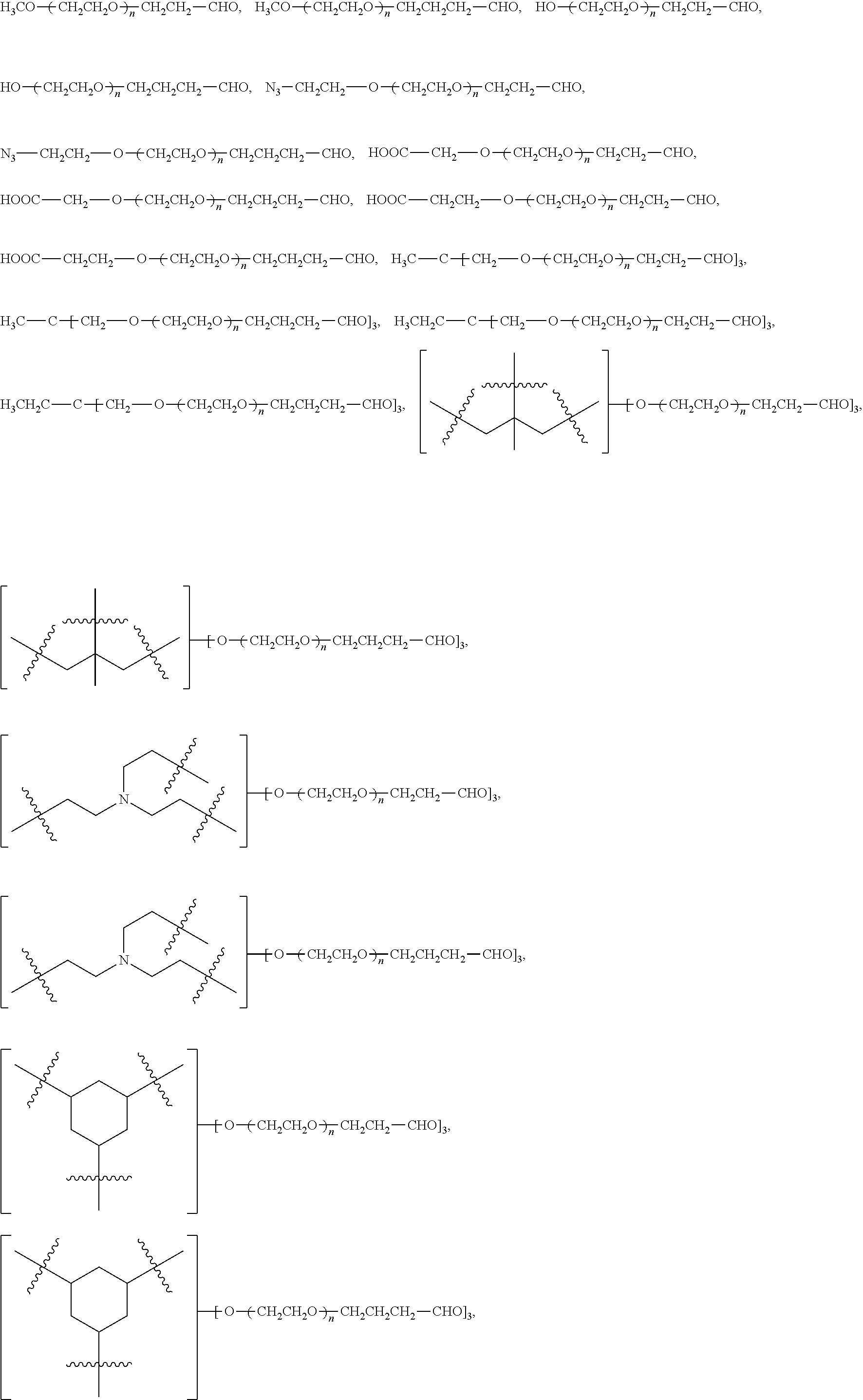

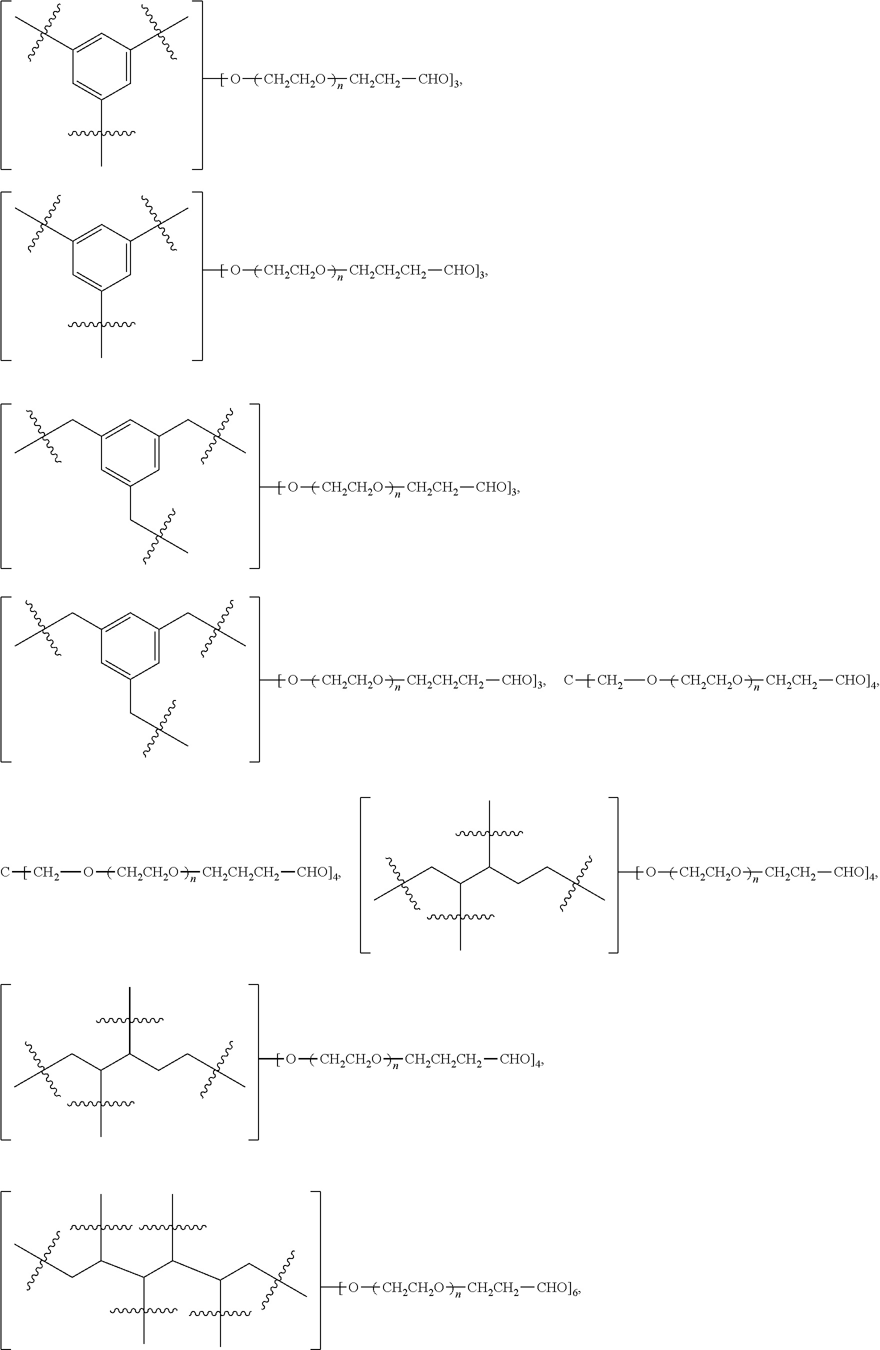

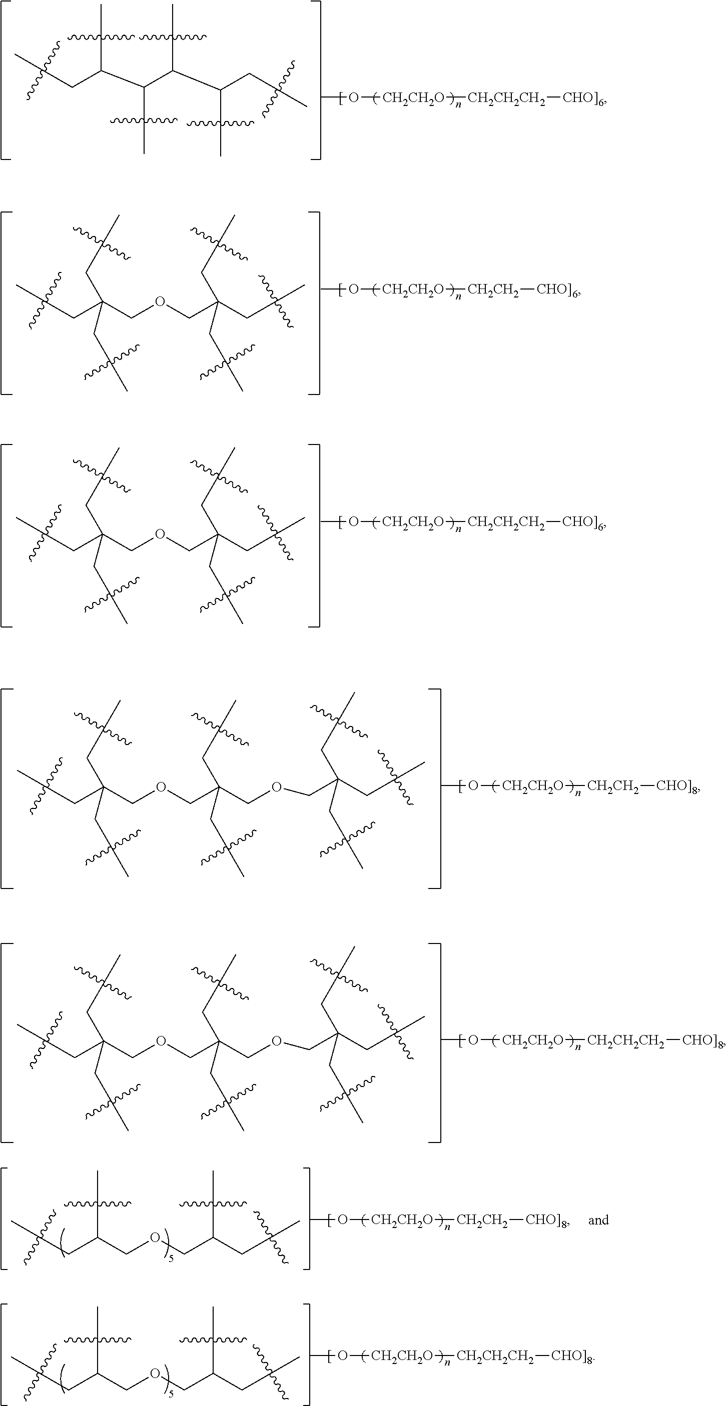

31. A method for preparing bio-related substance modified with a polyethylene glycol aldehyde derivative prepared according to claim 28, wherein, the bio-related substance is selected from the group consisting of a peptide, a polypeptide, a protein, a polysaccharide, a steroid, a nucleotide, an oligonucleotide, a polynucleotide, and a lipid.

32. The method for preparing bio-related substance according to claim 31, the bio-related substances contains an active amino group, or, after modification, contains at least one active amino group capable of coupling with the aldehyde group of the polyethylene glycol aldehyde derivative.
